(19) United States
(12) Patent Application Publication
(10) Pub. No.: US 2010/0278789 A1
(43) Pub. Date: Nov. 4, 2010

(54) METHODS OF GENERATING EXPANDED AND RE-DIFFERENTIATED ADULT ISLET BETA CELLS CAPABLE OF PRODUCING INSULIN

(75) Inventors: Shimon Efrat, Zikhron-Yaakov (IL); Yael Bar, Tel-Aviv (IL)

Correspondence Address: MARTIN D. MOYNIHAN d/b/a PRTSI, INC., P.O. BOX 16446, ARLINGTON, VA, 22215, US

(73) Assignee: Ramot At Tel Aviv University Ltd., Tel-Aviv (IL)

(21) Appl. No.: 12/817,223

(22) Filed: Jun. 17, 2010

Related U.S. Application Data
(63) Continuation-in-part of application No. PCT/IL2008/001624, filed on Dec. 16, 2008.
(60) Provisional application No. 61/006,109, filed on Dec. 19, 2007.

(51) Int. Cl. A61K 35/12; C12N 5/02; A61K 31/7088; C12N 5/071; C12Q 1/02

(52) U.S. Cl. 424/93.7; 435/377; 514/44 A; 435/372.2; 435/29

(57) ABSTRACT

A method of increasing insulin content in adult islet beta cells is disclosed. The method comprises contacting the adult islet beta cells with an agent capable of down-regulating activity and/or expression of at least one component participating in a NOTCH pathway, the component being up-regulated in beta cell dedifferentiation above a predetermined threshold, thereby increasing the insulin content in adult islet beta cells. Methods of labeling dedifferentiated adult islet beta cells are also disclosed. Cell populations generated using the methods of the present invention and uses thereof are also disclosed.

### RELATED APPLICATION/S

This application is a continuation-in-part (CIP) of PCT Patent Application No. PCT/IL2008/001624 filed Dec. 16, 2008, which claims the benefit of priority from U.S. Provisonal Patent Application No. 61/006,109 filed Dec. 19, 2007. The contents of all of the above applications are incorporated by reference as if fully set forth herein.

### FIELD AND BACKGROUND OF THE INVENTION

The present invention, in some embodiments thereof, relates to redifferentiated populations of expanded adult islet beta cells and, more particularly, but not exclusively, to agents capable of down-regulating the NOTCH pathway for the generation of same.

Beta-cell replacement by transplantation is a promising approach for treatment of type 1 diabetes, however its application on a large scale is limited by availability of pancreas donors. In a normal adult pancreas a slow rate of beta-cell renewal is responsible for maintenance of an adequate functional beta-cell mass. This rate is accelerated in conditions of increased demands for insulin, such as pregnancy and obesity. Work in an animal model demonstrated that new beta cells are generated in adult mice predominantly by replication of pre-existing beta cells, rather than by neogenesis from insulin-negative stem/progenitor cells. This finding has raised hopes for recapitulation of beta-cell expansion in cultures of adult human islets. However, previous attempts at in vitro expansion of adult human beta cells resulted in a limited number of cell population doublings and loss of insulin expression. Insulin-negative cells with a considerable proliferative capacity were derived from cultured human islets. Insulin expression in these cells could be induced by changing the culture conditions. One possible interpretation of these results is that beta cells survive, dedifferentiate, and divide in culture. Genetic cell-lineage tracing studies, in which cultured adult human islets were labeled, demonstrated that in contrast to mouse beta cells, dedifferentiated, label-positive cells derived from human beta cells could be induced to significantly proliferate in vitro (Russ HA, Bar Y, Ravassard P, Efrat S (2008) Diabetes 57:1575-1583). These cells may therefore be of value for development of cell therapy for type 1 diabetes, since they may retain some beta-cell-specific chromatin structure to allow their redifferentiation.

In the developing pancreas important cell-fate decisions, including the switch from proliferation to differentiation, and the choice between exocrine and endocrine fates, as well as among different endocrine fates, are regulated by the NOTCH signaling pathway. Expression of NOTCH ligands on a differentiating cell inhibits development of the same phenotype in neighboring cells, in a mechanism termed lateral inhibition. Ligand binding to NOTCH receptors on a neighboring cell results in cleavage of the NOTCH intracellular domain (NICD), which enters the nucleus and forms a complex that modulates gene expression. The Hairy and Enhancer of Split (HES) family of transcriptional regulators is a major target of the NICD complex. In fetal pancreas HES1 acts as an inhibitor of neurogenin 3 (NGN3) gene expression, which is required for islet development. In addition, HES1 regulates the cell cycle by inhibiting expression of genes encoding the cyclin kinase inhibitors p27 and p57. Some evidence suggests it may also inhibit insulin gene expression. Overall, HES1 is associated with promoting cell replication and preventing cell differentiation. Forced expression of NOTCH inhibits pancreas cell differentiation, while mice with null mutations in genes encoding NOTCH pathway components exhibit accelerated differentiation of endocrine pancreas. The NOTCH pathway is not normally expressed in the adult pancreas, however, it is activated in conditions associated with cell dedifferentiation and replication, such as regeneration following experimental pancreatitis, pancreatic neoplasia, metaplasia of cultured pancreatic exocrine cells, and in rat beta cells exposed to cytokines.

U.S. Pat. Appl. No. 20080014182 teaches method of redifferentiating expanded beta cells by differentiating same in a medium comprising betacellulin.

Additional background art includes Weinberg N, et al, (2007) Diabetes. 56(5): 1299-304.

### SUMMARY OF THE INVENTION

According to an aspect of some embodiments of the present invention there is provided a method of generating adult islet beta cells useful for the treatment of diabetes, the method comprising contacting the adult islet beta cells with an agent capable of down-regulating activity and/or expression of at least one component participating in a NOTCH pathway, the component being up-regulated in beta cell dedifferentiation above a predetermined threshold, thereby increasing the insulin content in adult islet beta cells.

According to some embodiments of the invention, the contacting is effected in a serum-free medium.

According to some embodiments of the invention, the method further comoprises incubating the adult islet beta cells in a culturing medium, thereby obtaining expanded adult islet beta cells prior to the contacting.

According to some embodiments of the invention, the increasing is effected in vivo.

According to some embodiments of the invention, the increasing is effected ex vivo.

According to an aspect of some embodiments of the present invention there is provided a method of treating diabetes in a subject, comprising

(a) contacting a population of expanded adult islet beta cells with an agent capable of down-regulating activity and/or expression of at least one component participating in a NOTCH pathway to generate a population of re-differentiated, expanded adult islet beta cells, the component being up-regulated in B cell dedifferentiation above a predetermined threshold; and

(b) transplanting a therapeutically effective amount of the population of re-differentiated, expanded adult islet beta cells into the subject, thereby treating diabetes.

According to an aspect of some embodiments of the present invention there is provided a method of purifying a population of dedifferentiated B cells, the method comprising:

(a) permanently tagging primary B cells of cultured human islets, wherein the tagging is irrespective of a subsequent differentiation status of the B cells, to generate a population of permanently tagged B cells;

(b) culturing the permanently tagged B cells under conditions sufficient to allow dedifferentiation of the tagged B cells to generate a population of dedifferentiated tagged B cells; and

(c) isolating the population of dedifferentiated tagged B cells, thereby purifying the population of dedifferentiated B cells.

According to an aspect of some embodiments of the present invention there is provided an isolated population of primary human dedifferentiated B cells, purified according to the method of the present invention.

According to an aspect of some embodiments of the present invention there is provided an isolated population of B cells generated by redifferentiating the isolated population of primary human dedifferentiated cells of the present invention.

According to an aspect of some embodiments of the present invention there is provided an isolated population of B cells, comprising a heterologous oligonucleotide capable of down-regulating an activity and/or expression of at least one component participating in a NOTCH pathway.

According to an aspect of some embodiments of the present invention there is provided a method of identifying an agent capable of affecting proliferation and/or redifferentiation of dedifferentiated B cells, the method comprising contacting the agent with the isolated population of cells of the present invention under conditions that allow redifferentiation and/or replication of the dedifferentiated B cells, wherein a change in replication and/or differentiation state of the isolated population of cells is indicative of an agent capable of affecting replication and/or redifferentiation of dedifferentiated B cells.

According to some embodiments of the invention, the agent is an oligonucleotide directed to an endogenous nucleic acid sequence expressing the at least one component participating in the NOTCH pathway.

According to some embodiments of the invention, the at least one component is selected from the group consisting of Hairy and Enhancer of Split 1 (HES1), NOTCH1, NOTCH 2 and NOTCH 3.

According to some embodiments of the invention, the at least one component is HES1.

According to some embodiments of the invention, the agent is an siRNA molecule as set forth in SEQ ID NO: 7, SEQ ID NO: 10 or SEQ ID NO: 15.

According to some embodiments of the invention, the agent is a gamma secretase inhibitor.

According to some embodiments of the invention, the adult islet beta cells are trypsinized.

According to some embodiments of the invention, the permanently tagging B cells is effected by transfecting the human islets with two expression constructs, wherein a first expression construct comprises a polynucleotide encoding a Cre recombinase polypeptide operatively linked to a B cell specific promoter; and wherein a second expression construct comprises a first polynucleotide encoding a first detectable moiety operatively linked to a constitutive promoter, the first polynucleotide being flanked by LoxP polynucleotides, the second expression construct further comprising a second polynucleotide encoding a second detectable moiety, the second polynucleotide being positioned 3′ to the first polynucleotide.

According to some embodiments of the invention, the first polynucleotide comprises a nucleic acid sequence as set forth in SEQ ID NO: 11.

According to some embodiments of the invention, the second polynucleotide comprises a nucleic acid sequence as set forth in SEQ ID NO: 12.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1A-E are graphs and photographs illustrating that upregulation of HES1 in cultured human islet cells and eGFP cells derived from beta cells correlates with downregulation of insulin. FIGS. 1A-B: qPCR analysis of RNA extracted from islet cells derived from 9 donors (each identified by a letter and digit code) at the indicated passage numbers. RQ, relative quantification compared to P0, which represents islet cells at culture initiation. Data are mean±SD (n=3). FIG. 1C: Immunoblotting for HES1 in protein extracted from islet cells at the indicated passage number. Beta-actin served as a loading control. FIGS. 1D-E: Immunofluorescence analysis of islet cells (left) and eGFP cells derived from beta cells (right) following 10 days in culture. FIG. 1D is merged with a phase contrast image. Arrow points to a beta cell which still expresses insulin and is not labeled for HES1. eGFP is detected in both cytoplasm and nucleus. Bar=20 μm.

FIGS. 2A-H are graphs and photographs illustrating the upregulation of the NOTCH pathway in cultured human islet cells and eGFP cells derived from beta cells. FIGS. 2A-F: qPCR analysis of RNA extracted from islet cells derived from 8 donors at the indicated passage numbers. RQ, relative quantification compared to P0. Data are mean±SD (n=3). FIGS. 2G-H: Immunofluorescence analysis of islet cells (FIG. 2G) and eGFP cells derived from beta cells (FIG. 2H) following 10 days in culture. Bar=20 μm.

FIGS. 3A-G are graphs and photographs illustrating downregulation of p57 in cultured human islet cells and eGFP⁺ cells derived from beta cells. FIG. 3A: qPCR analysis of RNA extracted from islet cells derived from 8 donors at the indicated passage numbers. RQ, relative quantification compared to P0. Data are mean±SD (n=3). FIGS. 3B-G: Immunofluorescence analysis of eGFP⁺ cells derived from beta cells following 10 days in culture. Solid arrow in FIG. 3D points to an eGFP⁺ cell which has lost both insulin and p57 expression; dashed arrow points to an eGFP⁺ beta cell which maintains both insulin and p57 expression. Arrow in FIG. 3G points to an eGFP⁺ cell which maintains p57 expression and is not labeled for Ki67. Bar=20 μm.

FIGS. 4A-F are graphs and photographs illustrating that prevention of HES1 upregulation by shRNA reduces replication of cultured human islet cells and eGFP⁺ cells derived from beta cells. FIG. 4A: Immunoblotting for HES1 in protein extracted from islet cells following infection with HES1 shRNA or non-target virus. FIG. 4B: incidence of BrdU⁺ cells among cultured islet cells following infection with HES1 shRNA or non-target virus. Data are mean±SD (n=3 donors; >1000 cells counted in culture from each donor; p<0.02). FIG. 4C: incidence of Ki67⁺ cells among eGFP⁺ cells from 2 representative donors following infection with HES1 shRNA or non-target virus. Data is based on >1000 cells counted in culture from each donor. FIG. 4D: Immunoblotting for PARP in protein extracted from islet cells following infection with non-target (lane 3) or HES1 shRNA virus (lane 4). Uninfected cells incubated with (lane 1) or without (lane 2) the apoptotic agent staurosporin served as controls. The lower band in lane 1 represents cleaved PARP. Beta-actin served as a loading control. FIGS. 4E-F: qPCR analysis of RNA extracted from islet cells following infection with HES1 shRNA or non-target virus. RQ, relative quantification compared to cells infected with non-target virus. Data are mean±SD (n=3 donors). Only the change in p57 is significant (p<0.04). All the analyses were done 14 days following viral infection.

FIGS. 5A-E are graphs and photographs illustrating that prevention of HES1 upregulation by shRNA reduces beta-cell dedifferentiation. FIG. 5A: qPCR analysis of RNA extracted from islet cells following infection with HES1 shRNA or non-target virus. RQ, relative quantification compared to cells infected with non-target virus. P0, islet cells at culture initiation. Data are mean±SD (n=3 donors). FIG. 5B: Incidence of insulin-positive cells among cultured islet cells following infection with HES1 shRNA or non-target virus. Data are mean ±SD (n=3 donors; >1000 cells counted in culture from each donor; p<0.016). FIG. 5C: Incidence of insulin-positive cells among eGFP⁺ beta cells from 2 representative donors following infection with HES1 shRNA or non-target virus. Data is based on >1000 cells counted in culture from each donor. FIGS. 5D-E: Immunofluorescence analysis of insulin in eGFP⁺ cells following infection with HES1 shRNA or non-target virus. Bar=100 μm. All the analyses were done 14 days following viral infection.

FIG. 6 is a photograph illustrating the results of an immunoblotting analyses for HES1 and p57 in human islet cells infected at p. 4 with HES1 shRNA or nontarget viruses. Cellular protein was extracted 9 days following infection and analyzed by immunoblotting with HES1 and p57 antibodies.

FIG. 7 is a bar graph illustrating quantitative RT-PCR analyses of RNA from human islet cells infected at p. 4 with HES1 shRNA or nontarget viruses. Cellular RNA was extracted 9 days following infection and analyzed with primers for the indicated genes. Values are mean±SD (n=4 donors), normalized to nontarget values (=1). Asterisks mark significant changes (p<0.01).

FIG. 8. Insulin content in human islet cells infected at p. 4 with HES1 shRNA or nontarget viruses. Cellular insulin was extracted 9 days following infection and analyzed by ELISA. Values are mean±SD (n=3 donors), normalized to nontarget values (=100%).

FIGS. 9A-F are photographs illustrating immunostaining for human C-peptide and p57 in human islet cells infected at p. 4 with HES1 shRNA or nontarget viruses. Cells were stained 9 days following infection. All nuclei were stained blue with DAPI. Bar=20 μm

FIG. 10 is bar graph quantifying the data of FIGS. 9A-F. >700 cells were counted in each group.

FIGS. 11A-B are photographs of the morphological changes in human islet cells infected at p. 4 with HES1 shRNA or nontarget viruses.

FIGS. 12A-B are schematic representation of the 2 lentivirus vectors. nls, nuclear localization signal.

FIGS. 13A-L are photographs illustrating the labeling of 293T cells with the 2-virus system. 293T cells were infected with the reporter virus alone, or in combination with a CMV-Cre or a RIP-Cre virus. Live cells were photographed 4 days following infection for DsRed2 (red) and eGFP (green) autofluorescence. All cells were visualized using a Nomarsky lens (left panels). Bar=100 μm.

FIGS. 14A-Q are photographs illustrating the labeling of □TC-tet cells with the 2-virus system. FIGS. 14A-H: Visualization of live cells. βTC-tet cells were infected with the reporter virus alone, or in combination with a RIP-Cre virus. Live cells were photographed 4 days following infection for DsRed2 (red) and eGFP (green) autofluorescence. All cells were visualized with phase contrast (left panels).Bar=100 μm. FIGS. 14I-L: Immunofluorescence analysis of □TC-tet cells infected with the RIP-Cre virus. Nuclei are stained blue with DAPI. Bar=10 μm. FIGS. 14J-Q: Immunostaining of βTC-tet cells infected with both viruses for insulin and eGFP. DsRed2+ cells are not seen in this field. Bar=10 μm.

FIGS. 15A-L are photographs illustrating the labeling of human islet cells with the 2-virus system. FIGS. 15A-H: Dissociated islet cells were infected with the reporter virus alone, or in combination with a RIP-Cre virus. Live cells were photographed at P7 for DsRed2 (red) and eGFP (green) autofluorescence. All cells were visualized with phase contrast (left panels). Bar=100 μm. FIGS. 15I-L: Islet cells infected with the RIP-Cre virus alone were stained 36 hours post infection with Cre and insulin antibodies. Nuclei were stained blue with DAPI. Bar=20 μm.

FIGS. 16A-H are graphs and photographs illustrating the specificity of beta-cell labeling in the mixed islet cell population. FIGS. 16A-B: The fraction of eGFP+ cells expressing individual islet hormones, CK19, or amylase on days 5-6 of the culture (4-5 days following viral infection) (top), and the fraction of cells positive for each protein which were labeled with eGFP (bottom). +D, cells incubated with diazoxide. Data are mean±SD (n=3), based on >1,000 cells counted for each protein from each of 3 donors. The inset on FIG. 16A shows data from a representative donor, based on >500 cells counted at each time point. Note that the data for the 3 non-beta cell hormones includes cells which were double-positive for insulin and the respective hormone; the latter are therefore also included in the fraction of eGFP+/ins+ cells. FIGS. 16C-H depict representative cells from the experiment in FIGS. 16A-B, immunostained for the indicated pancreatic proteins (blue) and eGFP (green). eGFP is detected in both cytoplasm and nucleus. Bar=10 μm.

FIGS. 17A-I are photographs and graphs illustrating replication of eGFP+ cells in mixed islet cell culture. FIGS. 17A-F: Immunostaining for Ki67 (magenta) and eGFP (green) in cells analyzed at the indicated passages. Bar=10 μm. FIG. 17G: incidence of eGFP+, DsRed2+, and unlabeled cells among all cells at the indicated passages, based on cell cytometry of 2-10×103 cells in each sample. Data are mean±SD (n=4 donors). FIGS. 17H-I: incidence of each cell type among all cells (FIG. 17H) and among Ki67+ cells (FIG. 171) in consecutive passages of cultured islet cells from a representative donor, based on >1,000 cells counted at each passage.

FIGS. 18A-F are photographs illustrating the labeling of mouse islet cells with the 2-virus system. FIGS. 18A-C: Dissociated islet cells were infected with the 2 viruses and analyzed 5 days post-infection for eGFP self-fluorescence and immunofluorescence with insulin antibodies. The figure shows a representative cell. FIGS. 18D-F: Labeled cells were analyzed 11 days post-infection for eGFP self-fluorescence and immunofluorescence with antibodies to mouse Ki67. Most eGFP⁺ cells did not stain for Ki67.

FIGS. 19A-H are photographs illustrating the analyses of FACS-sorted eGFP+ cells. FIGS. 19A-C: Mixed cell population at P12 (19A), and eGFP+ (19B) and DsRed2+ cells (19C) sorted at P8 and visualized by eGFP immunofluorescence (green) and DsRed2 self-fluorescence (red). Nuclei are stained blue with DAPI. All panels are merged from micrographs of the 3 individual colors. Bar=20 μm. FIG. 19D: PCR analysis of DNA from unsorted cells at P8 (lane 1), and DsRed2+ (lane 2) and eGFP+ (lane 3) cells sorted at the same passage, with primers for the reporter vector. Lane 4, uninfected cells. Lane 5, DNA ladder. The analysis was reproducible in cells from 3 donors, of which one is shown. FIG. 19E: Immunoblotting of protein from replicating cells at P10 with (lane 1) or without (lane 2) treatment with the apoptotic agent staurosporin, and from unsorted (lane 3), and DsRed2+ (lane 4) and eGFP+ (lane 5) cells sorted at P8, and analyzed at P16 with the indicated antibodies. FIGS. 19F-H: Immunostaining for Ki67 (magenta) and eGFP (green) in eGFP+ cells sorted at P5, propagated thereafter in the presence of conditioned medium, and analyzed at the indicated passages. Bar=10 μm.

FIG. 20 is a plasmid map of pTripRip400-nlscre-DeltaU3 (SEQ ID NO: 13).

FIG. 21 is a plasmid may of pTri CMV Lox-Red-Lox EGFP (SEQ ID NO: 14).

FIGS. 22A-C are graphs illustrating induction of beta-cell transcripts in dedifferentiated islet cells by treatment with HES1 shRNA and serum free medium (SFM). Adult human islet cells were labeled with eGFP, sorted at passage 2, and expanded for a total of 7 population doublings. They were then infected with HES1 or nontarget shRNA viruses, and 4 days later trypsinized and shifted to SFM. RNA was extracted following 4 more days and analyzed by qRT-PCR. Control, cells infected with nontarget shRNA virus. SH3, cells infected with HES1 shRNA virus.

FIGS. 23A-D are photographs illustrating morphological changes induced in sorted GFP+ cells by HES1 shRNA and SFM. A,B, cells at passage 7 in growth medium. C, cells following treatment with HES1 shRNA and SFM. D, immunofluorescence of islet cells at passage 4 treated with SFM for 8 days and stained with antibodies to GFP and human C-peptide. The image shows 3 cells co-staining for the two proteins. In cells counted from 3 donors 4.7±3.0% of GFP+ cells were C-pep+, compared with <0.1% in cells grown in medium with serum. 59.3±15.5% of all C-pep+ cells were GFP+, a fraction close to the labeling efficiency, demonstrating that virtually all C-pep+ cells emerged by redifferentiation of dedifferentiated beta cells.

FIG. 24 is a graph illustrating an increase in insulin expression following incubation in a medium comprising a gamma secretase inhibitor.

### DESCRIPTION OF THE PREFERRED EMBODIMENTS

The present invention relates to redifferentiated populations of expanded adult islet beta cells and, more particularly, but not exclusively, to agents capable of down-regulating the NOTCH pathway for the generation of same. The present invention can be used in cell replacement therapy in the treatment of insulin dependant diabetes.

The principles and operation of the expanded and re-differentiated isolated population of adult beta cells and methods of generating same according to the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Type I diabetes is caused by the autoimmune destruction of the pancreatic islet insulin-producing beta cells. Insulin administration does not prevent the long-term complications of the disease, since the optimal insulin dosage is difficult to adjust. Replacement of the damaged cells with regulated insulin-producing cells is considered the ultimate cure for type 1 diabetes. Pancreas transplantation has been successful but is severely limited by the shortage of donors. With the development of new islet isolation and immunosuppression procedures, significant success has been reported using islets from 2-3 donors per recipient (Shapiro A M, Lakey J R, Ryan E A et al. New Engl J Med 2000;343:230-238). This progress underscores the urgent need for developing alternatives to human pancreas donors, namely abundant sources of cultured human β cells for transplantation.

While reducing the present invention to practice, the present inventors have uncovered novel conditions for increasing insulin content in dedifferentiated expanded beta cells (i.e. re-differentiating cells). The present invention exploits these finding to provide a viable source of functioning beta cells for transplantation into diabetic patients. The present inventors have also uncovered a novel approach for purifying dedifferentiated beta cells from cultured islets, thereby allowing for a pure population of cells as a starting material for redifferentiation and for screening additional agents capable of redifferentiation.

As is illustrated hereinbelow and in the Examples section which follows the present inventor has uncovered that down-regulating components of the NOTCH pathway that are typically up-regulated during the beta cell dedifferentiation process promotes an increase in insulin content thereof.

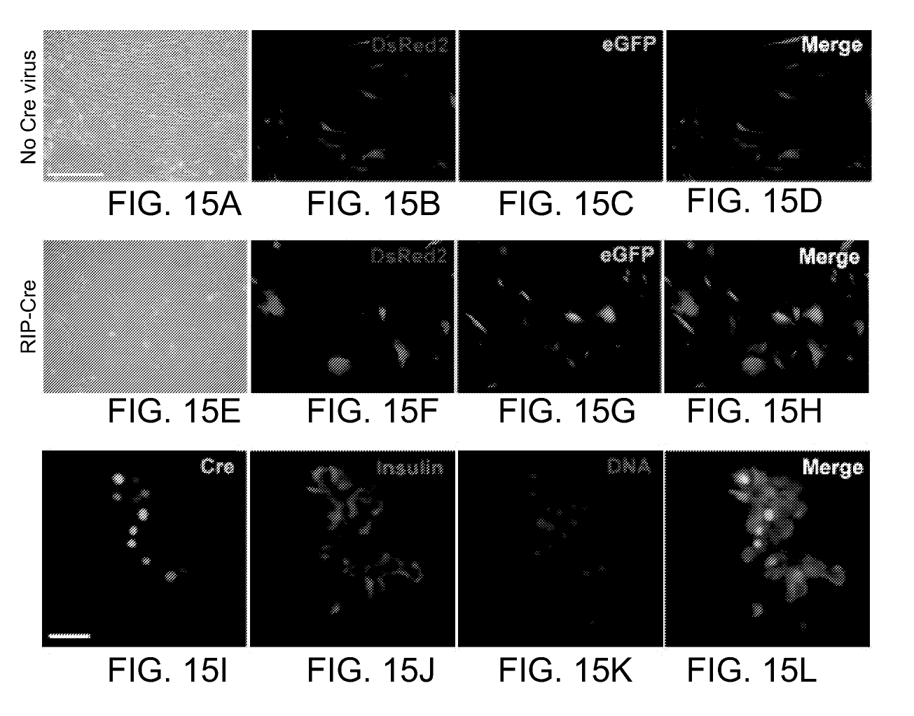

Whilst reducing the present invention to practice the present inventors used a genetic recombination approach to demonstrate the feasibility of cell-specific labeling of cultured primary human cells (FIGS. 15A-H). Using this method, the present inventors were able to isolate pure populations of dedifferentiated B cells.

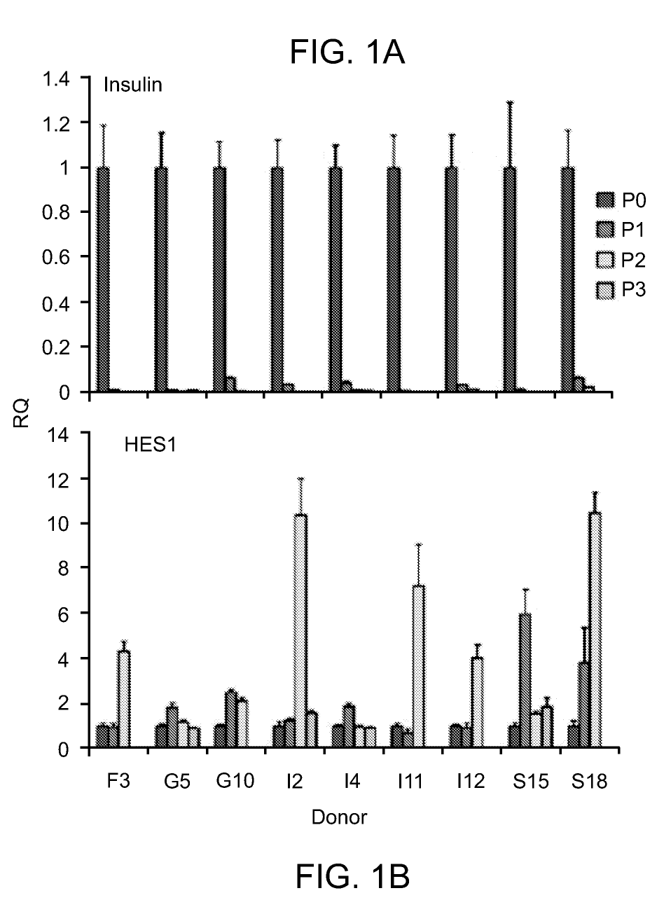

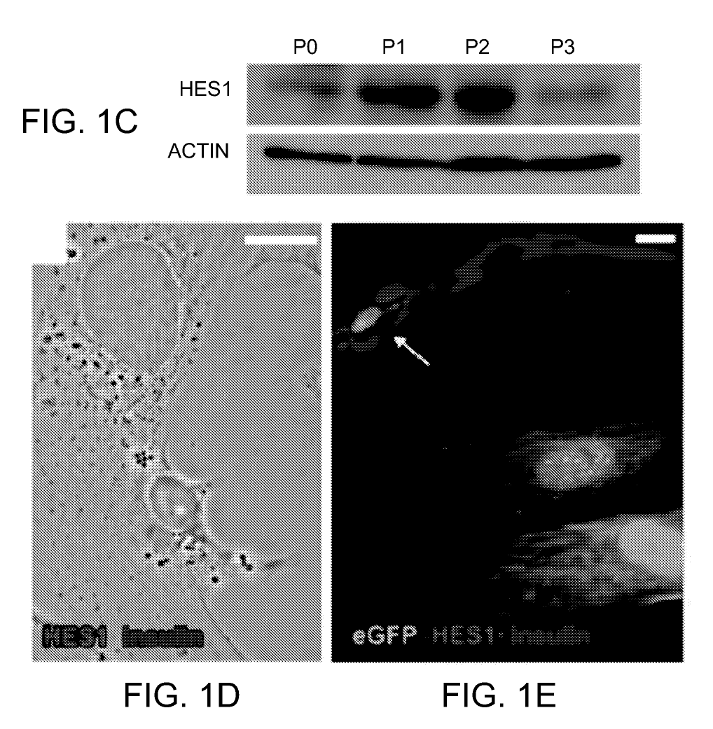

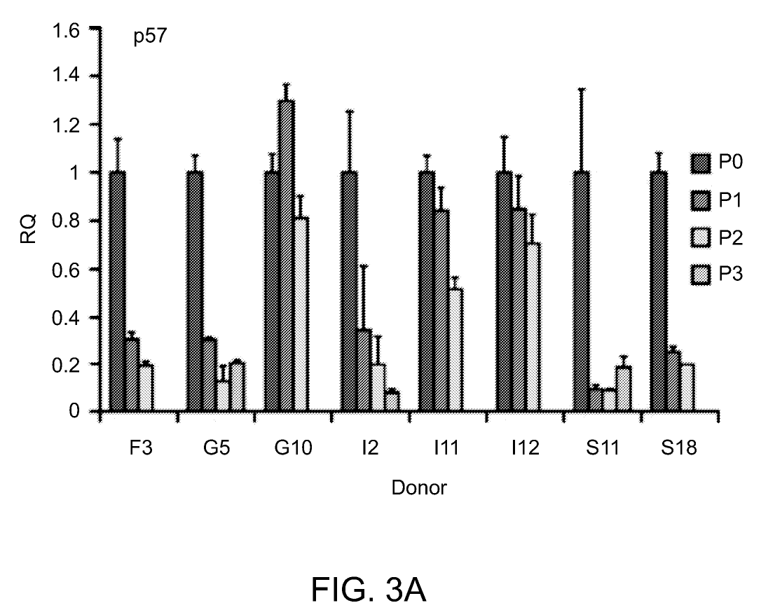

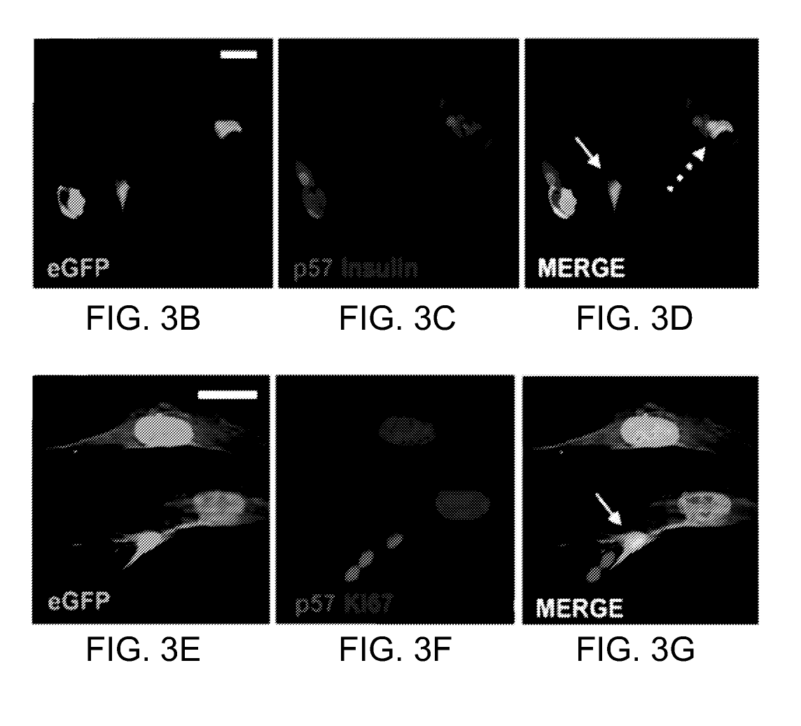

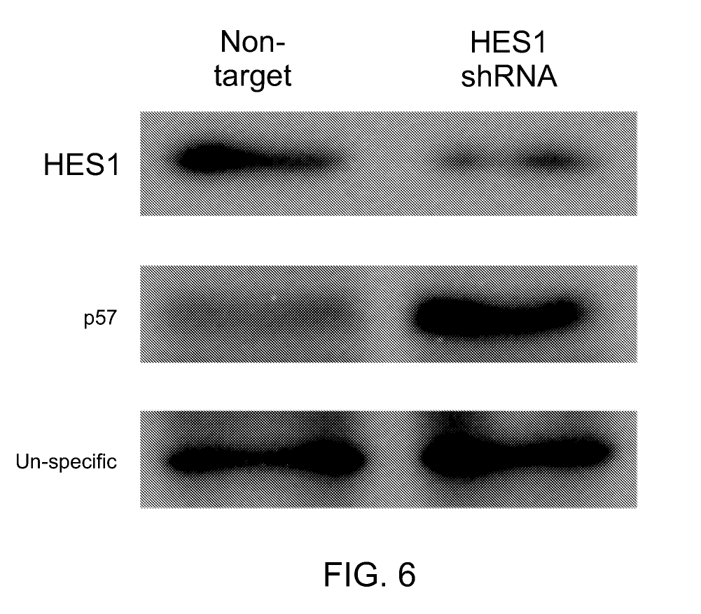

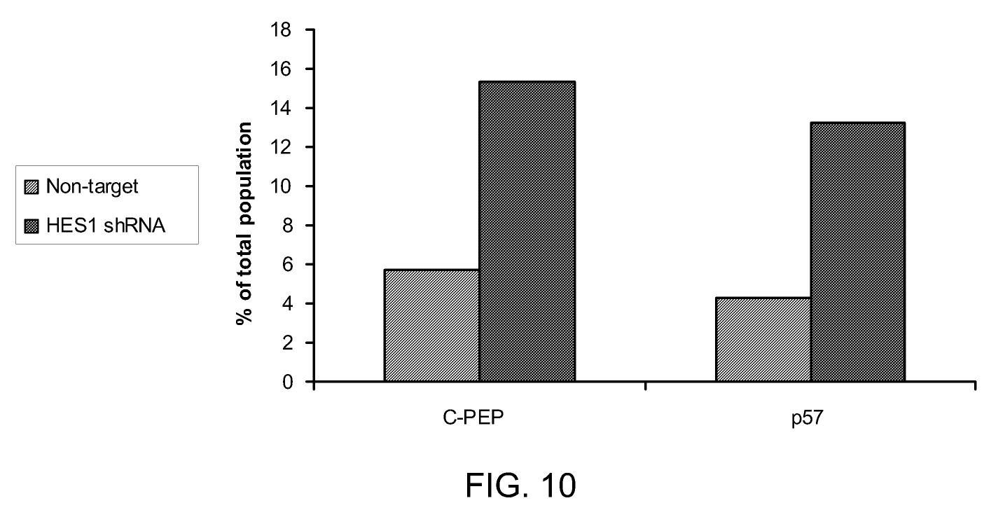

Whilst further reducing the present invention to practice, the present inventors have shown that Hairy and Enhancer of Split 1 (HES1), NOTCH1, NOTCH 2 and NOTCH 3 are all upregulated during the B cell dedifferentiation process (FIGS. 1A-B and 2A-F). The present inventors showed that inhibition of expression of one of these NOTCH pathway components (HES1) during the first 2 weeks of human islet cell culture, resulted in significantly reduced beta-cell replication and dedifferentiation (FIGS. 3A-G and 4A-F). In addition, the present inventors have demonstrated a reproducible differentiating effect of HES1 shRNA in dedifferentiated human islet cells expanded in culture, manifested by an increase in expression of the cell cycle inhibitor p57 and markers of beta-cell differentiation, most notably insulin (FIGS. 6-10). The present inventors conclude that agents capable of downregulating NOTCH pathway components (including HES1, NOTCH1, NOTCH 2 and/or NOTCH 3) can be used for redifferentiation of dedifferentiated human islet cells following expansion in culture.

Thus, according to one aspect of the present invention, there is provided a method of ex-vivo expanding and re-differentiating adult islet beta cells comprising:

(a) incubating adult islet beta cells in a culturing medium, thereby obtaining expanded adult islet beta cells; and

(b) contacting the expanded adult islet beta cells with an agent capable of down-regulating activity and/or expression of at least one component participating in a NOTCH pathway, the component being up-regulated in B cell dedifferentiation above a predetermined threshold;

thereby expanding and re-differentiating adult islet beta cells.

As used herein, the phrase “adult islet beta cells” refers to post-natal (e.g., non-embryonic) pancreatic islet endocrine cells which are capable of secreting insulin in response to elevated glucose concentrations and express typical beta cell markers. Examples of beta cell markers include, but are not limited to, insulin, pdx, Hnf3β, PC1/3, Beta2, Nkx2.2, GLUT2 and PC2.

The isolated adult islet beta cells of this aspect of the present invention may be of homogeneous or heterogeneous nature.

Thus, for example, the adult islet beta cells of this aspect of the present invention may be comprised in isolated pancreatic islets. Islet cells are typically comprised of the following: 1) beta cells that produce insulin; 2) alpha cells that produce glucagon; 3) delta cells (or D cells) that produce somatostatin; and/or F cells that produce pancreatic polypeptide. The polypeptide hormones (insulin, glucagon, somatostatin and pancreatic polypeptide) inside these cells are stored in secretary vesicles in the form of secretory granules.

Methods of isolating islets are well known in the art. For example, islets may be isolated from pancreatic tissue using collagenase and ficoll gradients. An exemplary method is described in U.S. Pat. Appl. No. 20080014182, incorporated herein by reference.

It will be appreciated that the beta cells may be purified from the islet at a later stage, following expansion but prior to redifferentiation (i.e. after they are dedifferentiated). Methods of purifying such cells are described herein below.

According to another embodiment, the adult islet beta cells of the present invention are dispersed into a single cell suspension—e.g. by the addition of trypsin or by trituration.

The adult islet beta cells may be further isolated being substantially free from other substances (e.g., other cells, proteins, nucleic acids, etc.) that are present in its in-vivo environment e.g. by FACs sorting.

The adult islet beta cells may be obtained from any autologous or non-autologous (i.e., allogeneic or xenogeneic) mammalian donor. For example, cells may be isolated from a human cadaver.

As used herein, the term “expanded adult islet beta cells” refers to B cells that have been increased in number by the process of cell division, rather than B cells enlarged by hypertrophy.

The present invention contemplates any medium for the culturing of the adult islet beta cells to obtain expanded adult islet beta. According to one embodiment, the medium is CMRL-1066.

As used herein, the term “CMRL 1066” refers to the serum free medium, originally developed by Connaught Medical Research Laboratories for the culture of L cells, and includes any other derivations thereof provided that the basic function of CMRL is preserved. CMRL-1060 medium is commercially available in either liquid or powder form from companies including Gibco BRL, Grand Island, N.Y., catalogue number 11530-037; Cell and Molecular Technologies, Phillipsburg N.J.; Biofluids Inc, Rockville, Md.; Bioreclamation Inc. East Meadow, N.Y.; United States Biological, Swampscott, Mass.; Sigma Chemical Company, St. Louis, Mo.; Cellgro/Mediatech, Herndon, Va. and Life technologies, Rockville Md.

The medium used to culture the beta cells may further comprise supplementary constituents which may improve growth and/or viability thereof. These include, but are not limited to, growth factors (e.g. hepatocyte growth factor, nerve growth factor and/or epidermal growth factor) serum (e.g. fetal calf serum or fetal bovine serum), glucose (e.g. 5.6 mM) and antibiotics.

Non-apoptotic culturing conditions for adult islet beta cells are known in the art—see for example U.S. Pat. Appl. No. 20080014182. According to one embodiment, beta cells are passaged every seven days and refed twice a week. According to the teachings of U.S. Pat. Appl. No. 20080014182, adult islet beta cells may be expanded 65,000 fold without any detectable apoptosis. According to another embodiment, the adult islet beta cells are propagated as anchorage-dependent cells by attaching to a solid substrate (i.e., a monolayer type of cell growth).

As mentioned hereinabove, following expansion, the adult islet beta cells are redifferentiated by contacting them with an agent capable of down-regulating activity and/or expression of at least one component participating in a NOTCH pathway, the component being up-regulated in B cell dedifferentiation above a predetermined threshold. According to one embodiment, the contacting is effected in serum free medium.

As used herein the term “re-differentiating” refers to the altering of a cell such that it passes from one of a less defined function to one of a more defined function (may also be referred to as more differentiated). For example, the defined functions of an adult beta cell include storing insulin and secreting insulin in response to glucose. Re-differentiation of the expanded adult islet beta cells of the present invention may include such processes as increasing beta cell insulin content, increasing sensitivity to glucose and/or increasing secretory apparatus. Methods of increasing beta cell insulin content may include increasing insulin transcription and/or post transcriptional control and/or increasing translation and/or post-translational control. Methods of increasing beta cell insulin content may also include enhancing insulin storage and/or retarding insulin breakdown. Methods of increasing sensitivity to glucose may include increasing the expression of glucose transporters.

The redifferentiating may be effected for 5 days-2 weeks, for example 8 days, 10 days or 12 days.

The phrase “component participating in the NOTCH pathway” refers to a polypeptide or polynucleotide involved in the NOTCH signaling pathway. Exemplary components are described herein below.

The Notch signaling pathway is a conserved intercellular signaling mechanism that is essential for proper embryonic development in numerous metazoan organisms.

Members of the Notch gene family (NOTCHs) encode transmembrane receptors that are critical for various cell fate decisions. Multiple ligands that activate Notch and related receptors have been identified, including Senate and Delta in Drosophila and JAG1 (MIM.601920) in vertebrates.

Four different Notch receptors (NOTCHs: NOTCH1 to NOTCH4) and five ligands (Jagged-1 (JAG1) and -2 (JAG2) and Delta-like [DLLs]: DLL1, DLL2 and DLL4) have been characterized in mammalian cells. These transmembrane receptors and ligands are expressed in different combinations in most, if not all, cell types. The Notch pathway regulates cell fate determination of neighbouring cells through lateral inhibitiona, depending on their ability to express either the receptors or the ligands.

Following ligand binding, NOTCHs are activated by a series of cleavages that releases its intracellular domain (NICD). This processing requires the activity of two proteases, namely ADAM17 (tumour necrosis factor-α converting enzyme or TACE MIM.603369) and presenilin-1 (PSEN1 MIM.104311), both of which also fall under the category of a component of a NOTCH pathway.

Nuclear translocation of NICD results in transcriptional activation of genes of the HESs family (Hes/E(sp1) family) and HEYs family (Hesr/Hey family) through interaction of NICD with RBPSUH (or CBF1 MIM.147183), Su(H), and Lag-1, which is also known as the recombination signal sequence-binding protein (RBP)-j (also called Suppressor of Hairless, Su(H)), each of these also falling under the category of a component of a NOTCH pathway.

Overall, when activated, Notch signalling enables neighbouring cells to acquire distinct phenotypes, through a process named lateral inhibition. The Notch receptor is pre-cleaved in the Golgi and is targeted subsequently to the plasma membrane where it interacts with ligands located on neighbouring cells. Receptor—ligand interaction results in a conformational change in the receptor, thus enabling additional cleavages by TACE and the γ-secretase complex. This proteolytic activity enables the Notch intracellular domain (NICD) to translocate to the nucleus where it activates the transcription of target genes (e.g. the Hes and Hey family of transcriptional repressors).

Monoubiquitylation (Ub) of the ligand by mindbomb (MIB) induces endocytosis of the ligand and the Notch extracellular domain (NECD) into the ligand cells where additional signalling might be initiated.

Notch receptors undergo a complex set of proteolytic processing events in response to ligand activating, which eventuallyleads to release of the intracellular domain of the receptor. Signal transduction is normally initiated by binding to transmembrane ligands of the Serrate or Delta class, which induces proteolytic release of the intracellular NOTCH domain (NICD).

Free NICD translocates to the nucleus to form a short-lived complex with a Rel-like transcription factor, CSL, and Mastermind-like co-activators that activates lineage-specific programs of gene expression.

As mentioned, the present invention contemplates down-regulating any component of the NOTCH pathway that is up-regulated in B cell dedifferentiation above a predetermined threshold.

Methods of analyzing whether a particular component is upregulated during B cell differentiation are known in the art, and may be effected on the RNA level (using techniques such as Northern blot analysis, RT-PCR and oligonucleotides microarray) and/or the protein level (using techniques such as ELISA, Western blot analysis, immunohistochemistry and the like, which may be effected using antibodies specific to the NOTCH pathway component).

According to one embodiment the NOTCH pathway component is upregulated by at least 1.5 times, more preferably by at least 2 times and more preferably by at least 3 times.

According to another embodiment, the NOTCH pathway component is Hairy and Enhancer of Split 1 (HES1; NM_{—}005524, NP_{—}005515), NOTCH1 (NM_{—}017617, NP_{—}060087.3) NOTCH 2 (NM_{—}024408, NP_{—}077719.2)and NOTCH 3 (NM_{—}000435, NP_{—}000426 .2).

Downregulation of NOTCH pathway components can be effected on the genomic and/or the transcript level using a variety of molecules which interfere with transcription and/or translation (e.g., RNA silencing agents, Ribozyme, DNAzyme and antisense), or on the protein level using e.g., antagonists, enzymes that cleave the polypeptide and the like.

Following is a list of agents capable of downregulating expression level and/or activity of NOTCH pathway components.

One example, of an agent capable of downregulating a NOTCH pathway component is an antibody or antibody fragment capable of specifically binding thereto. Preferably, the antibody is capable of being internalized by the cell and entering the nucleus.

The term “antibody” as used in this invention includes intact molecules as well as functional fragments thereof, such as Fab, F(ab′)2, and Fv that are capable of binding to macrophages. These functional antibody fragments are defined as follows: (1) Fab, the fragment which contains a monovalent antigen-binding fragment of an antibody molecule, can be produced by digestion of whole antibody with the enzyme papain to yield an intact light chain and a portion of one heavy chain; (2) Fab′, the fragment of an antibody molecule that can be obtained by treating whole antibody with pepsin, followed by reduction, to yield an intact light chain and a portion of the heavy chain; two Fab′ fragments are obtained per antibody molecule; (3) (Fab′)2, the fragment of the antibody that can be obtained by treating whole antibody with the enzyme pepsin without subsequent reduction; F(ab′)2 is a dimer of two Fab′ fragments held together by two disulfide bonds; (4) Fv, defined as a genetically engineered fragment containing the variable region of the light chain and the variable region of the heavy chain expressed as two chains; and (5) Single chain antibody (“SCA”), a genetically engineered molecule containing the variable region of the light chain and the variable region of the heavy chain, linked by a suitable polypeptide linker as a genetically fused single chain molecule.

Downregulation of a NOTCH pathway component can be also achieved by RNA silencing. As used herein, the phrase “RNA silencing” refers to a group of regulatory mechanisms [e.g. RNA interference (RNAi), transcriptional gene silencing (TGS), post-transcriptional gene silencing (PTGS), quelling, co-suppression, and translational repression] mediated by RNA molecules which result in the inhibition or “silencing” of the expression of a corresponding protein-coding gene. RNA silencing has been observed in many types of organisms, including plants, animals, and fungi.

As used herein, the term “RNA silencing agent” refers to an RNA which is capable of inhibiting or “silencing” the expression of a target gene. In certain embodiments, the RNA silencing agent is capable of preventing complete processing (e.g, the full translation and/or expression) of an mRNA molecule through a post-transcriptional silencing mechanism. RNA silencing agents include noncoding RNA molecules, for example RNA duplexes comprising paired strands, as well as precursor RNAs from which such small non-coding RNAs can be generated. Exemplary RNA silencing agents include dsRNAs such as siRNAs, miRNAs and shRNAs. In one embodiment, the RNA silencing agent is capable of inducing RNA interference. In another embodiment, the RNA silencing agent is capable of mediating translational repression.

RNA interference refers to the process of sequence-specific post-transcriptional gene silencing in animals mediated by short interfering RNAs (siRNAs). The corresponding process in plants is commonly referred to as post-transcriptional gene silencing or RNA silencing and is also referred to as quelling in fungi. The process of post-transcriptional gene silencing is thought to be an evolutionarily-conserved cellular defense mechanism used to prevent the expression of foreign genes and is commonly shared by diverse flora and phyla. Such protection from foreign gene expression may have evolved in response to the production of double-stranded RNAs (dsRNAs) derived from viral infection or from the random integration of transposon elements into a host genome via a cellular response that specifically destroys homologous single-stranded RNA or viral genomic RNA.

The presence of long dsRNAs in cells stimulates the activity of a ribonuclease III enzyme referred to as dicer. Dicer is involved in the processing of the dsRNA into short pieces of dsRNA known as short interfering RNAs (siRNAs). Short interfering RNAs derived from dicer activity are typically about 21 to about 23 nucleotides in length and comprise about 19 base pair duplexes. The RNAi response also features an endonuclease complex, commonly referred to as an RNA-induced silencing complex (RISC), which mediates cleavage of single-stranded RNA having sequence complementary to the antisense strand of the siRNA duplex. Cleavage of the target RNA takes place in the middle of the region complementary to the antisense strand of the siRNA duplex.

Accordingly, the present invention contemplates use of dsRNA to downregulate protein expression from mRNA.

According to one embodiment, the dsRNA is greater than 30 bp. The use of long dsRNAs (i.e. dsRNA greater than 30 bp) has been very limited owing to the belief that these longer regions of double stranded RNA will result in the induction of the interferon and PKR response. However, the use of long dsRNAs can provide numerous advantages in that the cell can select the optimal silencing sequence alleviating the need to test numerous siRNAs; long dsRNAs will allow for silencing libraries to have less complexity than would be necessary for siRNAs; and, perhaps most importantly, long dsRNA could prevent viral escape mutations when used as therapeutics.

Various studies demonstrate that long dsRNAs can be used to silence gene expression without inducing the stress response or causing significant off-target effects—see for example [Strat et al., Nucleic Acids Research, 2006, Vol. 34, No. 13 3803-3810; Bhargava A et al. Brain Res. Protoc. 2004;13:115-125; Diallo M., et al., Oligonucleotides. 2003;13:381-392; Paddison P. J., et al., Proc. Natl Acad. Sci. USA. 2002;99:1443-1448; Tran N., et al., FEBS Lett. 2004;573:127-134].

In particular, the present invention also contemplates introduction of long dsRNA (over 30 base transcripts) for gene silencing in cells where the interferon pathway is not activated (e.g. embryonic cells and oocytes) see for example Billy et al., PNAS 2001, Vol 98, pages 14428-14433. and Diallo et al, Oligonucleotides, Oct. 1, 2003, 13(5): 381-392. doi:10.1089/154545703322617069.

The present invention also contemplates introduction of long dsRNA specifically designed not to induce the interferon and PKR pathways for down-regulating gene expression. For example, Shinagwa and Ishii [Genes & Dev. 17 (11): 1340-1345, 2003] have developed a vector, named pDECAP, to express long double-strand RNA from an RNA polymerase II (Pol II) promoter. Because the transcripts from pDECAP lack both the 5′-cap structure and the 3′-poly(A) tail that facilitate ds-RNA export to the cytoplasm, long ds-RNA from pDECAP does not induce the interferon response.

Another method of evading the interferon and PKR pathways in mammalian systems is by introduction of small inhibitory RNAs (siRNAs) either via transfection or endogenous expression.

The term “siRNA” refers to small inhibitory RNA duplexes (generally between 18-30 basepairs) that induce the RNA interference (RNAi) pathway. Typically, siRNAs are chemically synthesized as 21 mers with a central 19 by duplex region and symmetric 2-base 3′-overhangs on the termini, although it has been recently described that chemically synthesized RNA duplexes of 25-30 base length can have as much as a 100-fold increase in potency compared with 21 mers at the same location. The observed increased potency obtained using longer RNAs in triggering RNAi is theorized to result from providing Dicer with a substrate (27 mer) instead of a product (21 mer) and that this improves the rate or efficiency of entry of the siRNA duplex into RISC.

It has been found that position of the 3′-overhang influences potency of an siRNA and asymmetric duplexes having a 3′-overhang on the antisense strand are generally more potent than those with the 3′-overhang on the sense strand (Rose et al., 2005). This can be attributed to asymmetrical strand loading into RISC, as the opposite efficacy patterns are observed when targeting the antisense transcript.

The strands of a double-stranded interfering RNA (e.g., an siRNA) may be connected to form a hairpin or stem-loop structure (e.g., an shRNA). Thus, as mentioned the RNA silencing agent of the present invention may also be a short hairpin RNA (shRNA).

The term “shRNA”, as used herein, refers to an RNA agent having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region. The number of nucleotides in the loop is a number between and including 3 to 23, or 5 to 15, or 7 to 13, or 4 to 9, or 9 to 11. Some of the nucleotides in the loop can be involved in base-pair interactions with other nucleotides in the loop. Examples of oligonucleotide sequences that can be used to form the loop include 5′-UUCAAGAGA-3′ (SEQ ID NO: 8; Brummelkamp, T. R. et al. (2002) Science 296: 550) and 5′-UUUGUGUAG-3′ (SEQ ID NO: 9; Castanotto, D. et al. (2002) RNA 8:1454). It will be recognized by one of skill in the art that the resulting single chain oligonucleotide forms a stem-loop or hairpin structure comprising a double-stranded region capable of interacting with the RNAi machinery.

According to another embodiment the RNA silencing agent may be a miRNA. miRNAs are small RNAs made from genes encoding primary transcripts of various sizes. They have been identified in both animals and plants. The primary transcript (termed the “pri-miRNA”) is processed through various nucleolytic steps to a shorter precursor miRNA, or “pre-miRNA.” The pre-miRNA is present in a folded form so that the final (mature) miRNA is present in a duplex, the two strands being referred to as the miRNA (the strand that will eventually basepair with the target) The pre-miRNA is a substrate for a form of dicer that removes the miRNA duplex from the precursor, after which, similarly to siRNAs, the duplex can be taken into the RISC complex. It has been demonstrated that miRNAs can be transgenically expressed and be effective through expression of a precursor form, rather than the entire primary form (Parizotto et al. (2004) Genes & Development 18:2237-2242 and Guo et al. (2005) Plant Cell 17:1376-1386).

Unlike, siRNAs, miRNAs bind to transcript sequences with only partial complementarity (Zeng et al., 2002, Molec. Cell 9:1327-1333) and repress translation without affecting steady-state RNA levels (Lee et al., 1993, Cell 75:843-854; Wightman et al., 1993, Cell 75:855-862). Both miRNAs and siRNAs are processed by Dicer and associate with components of the RNA-induced silencing complex (Hutvagner et al., 2001, Science 293:834-838; Grishok et al., 2001, Cell 106: 23-34; Ketting et al., 2001, Genes Dev. 15:2654-2659; Williams et al., 2002, Proc. Natl. Acad. Sci. USA 99:6889-6894; Hammond et al., 2001, Science 293:1146-1150; Mourlatos et al., 2002, Genes Dev. 16:720-728). A recent report (Hutvagner et al., 2002, Sciencexpress 297:2056-2060) hypothesizes that gene regulation through the miRNA pathway versus the siRNA pathway is determined solely by the degree of complementarity to the target transcript. It is speculated that siRNAs with only partial identity to the mRNA target will function in translational repression, similar to an miRNA, rather than triggering RNA degradation.

Synthesis of RNA silencing agents suitable for use with the present invention can be effected as follows. First, the NOTCH pathway component mRNA sequence is scanned downstream of the AUG start codon for AA dinucleotide sequences. Occurrence of each AA and the 3′ adjacent 19 nucleotides is recorded as potential siRNA target sites. Preferably, siRNA target sites are selected from the open reading frame, as untranslated regions (UTRs) are richer in regulatory protein binding sites. UTR-binding proteins and/or translation initiation complexes may interfere with binding of the siRNA endonuclease complex [Tuschl ChemBiochem. 2:239-245]. It will be appreciated though, that siRNAs directed at untranslated regions may also be effective, as demonstrated for GAPDH wherein siRNA directed at the 5′ UTR mediated about 90% decrease in cellular GAPDH mRNA and completely abolished protein level (www.ambion.com/techlib/tn/91/912.html).

Second, potential target sites are compared to an appropriate genomic database (e.g., human, mouse, rat etc.) using any sequence alignment software, such as the BLAST software available from the NCBI server (www.ncbi.nlm.nih.gov/BLAST/). Putative target sites which exhibit significant homology to other coding sequences are filtered out.

Qualifying target sequences are selected as template for siRNA synthesis. Preferred sequences are those including low G/C content as these have proven to be more effective in mediating gene silencing as compared to those with G/C content higher than 55%. Several target sites are preferably selected along the length of the target gene for evaluation. For better evaluation of the selected siRNAs, a negative control is preferably used in conjunction. Negative control siRNA preferably include the same nucleotide composition as the siRNAs but lack significant homology to the genome. Thus, a scrambled nucleotide sequence of the siRNA is preferably used, provided it does not display any significant homology to any other gene.

For example, a suitable siRNA capable of downregulating HES1 can be the siRNA of SEQ ID NO: 7 (TGGCCAGTTTGCTTTCCTCAT), of SEQ ID NO: 10 (CCAGATCAATGCCATGACCTA) or SEQ ID NO: 15 (GAAAGTCATCAAAGCCTATTA).

It will be appreciated that the RNA silencing agent of the present invention need not be limited to those molecules containing only RNA, but further encompasses chemically-modified nucleotides and non-nucleotides.

In some embodiments, the RNA silencing agent provided herein can be functionally associated with a cell-penetrating peptide.” As used herein, a “cell-penetrating peptide” is a peptide that comprises a short (about 12-30 residues) amino acid sequence or functional motif that confers the energy-independent (i.e., non-endocytotic) translocation properties associated with transport of the membrane-permeable complex across the plasma and/or nuclear membranes of a cell. The cell-penetrating peptide used in the membrane-permeable complex of the present invention preferably comprises at least one non-functional cysteine residue, which is either free or derivatized to form a disulfide link with a double-stranded ribonucleic acid that has been modified for such linkage. Representative amino acid motifs conferring such properties are listed in U.S. Pat. No. 6,348,185, the contents of which are expressly incorporated herein by reference. The cell-penetrating peptides of the present invention preferably include, but are not limited to, penetratin, transportan, pIsl, TAT(48-60), pVEC, MTS, and MAP.

Another agent capable of downregulating a NOTCH pathway component is a DNAzyme molecule capable of specifically cleaving an mRNA transcript or DNA sequence of the NOTCH pathway component. DNAzymes are single-stranded polynucleotides which are capable of cleaving both single and double stranded target sequences (Breaker, R. R. and Joyce, G. Chemistry and Biology 1995;2:655; Santoro, S. W. & Joyce, G. F. Proc. Natl, Acad. Sci. USA 1997;943:4262) A general model (the “10-23” model) for the DNAzyme has been proposed. “10-23” DNAzymes have a catalytic domain of 15 deoxyribonucleotides, flanked by two substrate-recognition domains of seven to nine deoxyribonucleotides each. This type of DNAzyme can effectively cleave its substrate RNA at purine:pyrimidine junctions (Santoro, S. W. & Joyce, G. F. Proc. Natl, Acad. Sci. USA 199; for rev of DNAzymes see Khachigian, L M [Curr Opin Mol Ther 4:119-21 (2002)].

Examples of construction and amplification of synthetic, engineered DNAzymes recognizing single and double-stranded target cleavage sites have been disclosed in U.S. Pat. No. 6,326,174 to Joyce et al. DNAzymes of similar design directed against the human Urokinase receptor were recently observed to inhibit Urokinase receptor expression, and successfully inhibit colon cancer cell metastasis (Itoh et al , 20002, Abstract 409, Ann Meeting Am Soc Gen Ther wwwdotasgtdotorg). In another application, DNAzymes complementary to bcr-ab1 oncogenes were successful in inhibiting the oncogenes expression in leukemia cells, and lessening relapse rates in autologous bone marrow transplant in cases of CML and ALL.

Downregulation of a NOTCH pathway component can also be effected by using an antisense polynucleotide capable of specifically hybridizing with an mRNA transcript encoding the NOTCH pathway component.

Design of antisense molecules which can be used to efficiently downregulate a NOTCH pathway component must be effected while considering two aspects important to the antisense approach. The first aspect is delivery of the oligonucleotide into the cytoplasm of the appropriate cells, while the second aspect is design of an oligonucleotide which specifically binds the designated mRNA within cells in a way which inhibits translation thereof.

The prior art teaches of a number of delivery strategies which can be used to efficiently deliver oligonucleotides into a wide variety of cell types [see, for example, Luft J Mol Med 76: 75-6 (1998); Kronenwett et al. Blood 91: 852-62 (1998); Rajur et al. Bioconjug Chem 8: 935-40 (1997); Lavigne et al. Biochem Biophys Res Commun 237: 566-71 (1997) and Aoki et al. (1997) Biochem Biophys Res Commun 231: 540-5 (1997)].

In addition, algorithms for identifying those sequences with the highest predicted binding affinity for their target mRNA based on a thermodynamic cycle that accounts for the energetics of structural alterations in both the target mRNA and the oligonucleotide are also available [see, for example, Walton et al. Biotechnol Bioeng 65: 1-9 (1999)].

Such algorithms have been successfully used to implement an antisense approach in cells. For example, the algorithm developed by Walton et al. enabled scientists to successfully design antisense oligonucleotides for rabbit beta-globin (RBG) and mouse tumor necrosis factor-alpha (TNF alpha) transcripts. The same research group has more recently reported that the antisense activity of rationally selected oligonucleotides against three model target mRNAs (human lactate dehydrogenase A and B and rat gp130) in cell culture as evaluated by a kinetic PCR technique proved effective in almost all cases, including tests against three different targets in two cell types with phosphodiester and phosphorothioate oligonucleotide chemistries.

In addition, several approaches for designing and predicting efficiency of specific oligonucleotides using an in vitro system were also published (Matveeva et al., Nature Biotechnology 16: 1374-1375 (1998)].

Another agent capable of downregulating a NOTCH pathway component is a ribozyme molecule capable of specifically cleaving an mRNA transcript encoding a NOTCH pathway component. Ribozymes are being increasingly used for the sequence-specific inhibition of gene expression by the cleavage of mRNAs encoding proteins of interest [Welch et al., Curr Opin Biotechnol. 9:486-96 (1998)]. The possibility of designing ribozymes to cleave any specific target RNA has rendered them valuable tools in both basic research and therapeutic applications. In the therapeutics area, ribozymes have been exploited to target viral RNAs in infectious diseases, dominant oncogenes in cancers and specific somatic mutations in genetic disorders [Welch et al., Clin Diagn Virol. 10:163-71 (1998)]. Most notably, several ribozyme gene therapy protocols for HIV patients are already in Phase 1 trials. More recently, ribozymes have been used for transgenic animal research, gene target validation and pathway elucidation. Several ribozymes are in various stages of clinical trials. ANGIOZYME was the first chemically synthesized ribozyme to be studied in human clinical trials. ANGIOZYME specifically inhibits formation of the VEGF-r (Vascular Endothelial Growth Factor receptor), a key component in the angiogenesis pathway. Ribozyme Pharmaceuticals, Inc., as well as other firms have demonstrated the importance of anti-angiogenesis therapeutics in animal models. HEPTAZYME, a ribozyme designed to selectively destroy Hepatitis C Virus (HCV) RNA, was found effective in decreasing Hepatitis C viral RNA in cell culture assays (Ribozyme Pharmaceuticals, Incorporated—WEB home page).

An additional method of regulating the expression of a NOTCH pathway component gene in cells is via triplex forming oligonuclotides (TFOs). Recent studies have shown that TFOs can be designed which can recognize and bind to polypurine/polypirimidine regions in double-stranded helical DNA in a sequence-specific manner. These recognition rules are outlined by Maher III, L. J., et al., Science, 1989;245:725-730; Moser, H. E., et al., Science,1987;238:645-630; Beal, P. A., et al, Science, 1992;251:1360-1363; Cooney, M., et al., Science, 1988;241:456-459; and Hogan, M. E., et al., EP Publication 375408. Modification of the oligonuclotides, such as the introduction of intercalators and backbone substitutions, and optimization of binding conditions (pH and cation concentration) have aided in overcoming inherent obstacles to TFO activity such as charge repulsion and instability, and it was recently shown that synthetic oligonucleotides can be targeted to specific sequences (for a recent review see Seidman and Glazer, J Clin Invest 2003;112:487-94).

In general, the triplex-forming oligonucleotide has the sequence correspondence:

| | | | | |
|---|---|---|---|---|
| oligo | 3′--A | G | G | T |
| duplex | 5′--A | G | C | T |
| duplex | 3′--T | C | G | A |

However, it has been shown that the A-AT and G-GC triplets have the greatest triple helical stability (Reither and Jeltsch, BMC Biochem, 2002, Sep. 12, Epub). The same authors have demonstrated that TFOs designed according to the A-AT and G-GC rule do not form non-specific triplexes, indicating that the triplex formation is indeed sequence specific.

Thus for any given sequence in the NOTCH pathway component regulatory region a triplex forming sequence may be devised. Triplex-forming oligonucleotides preferably are at least 15, more preferably 25, still more preferably 30 or more nucleotides in length, up to 50 or 100 bp.

Transfection of cells (for example, via cationic liposomes) with TFOs, and formation of the triple helical structure with the target DNA induces steric and functional changes, blocking transcription initiation and elongation, allowing the introduction of desired sequence changes in the endogenous DNA and resulting in the specific downregulation of gene expression. Examples of such suppression of gene expression in cells treated with TFOs include knockout of episomal supFG1 and endogenous HPRT genes in mammalian cells (Vasquez et al., Nucl Acids Res. 1999;27:1176-81, and Puri, et al, J Biol Chem, 2001;276:28991-98), and the sequence- and target specific downregulation of expression of the Ets2 transcription factor, important in prostate cancer etiology (Carbone, et al, Nucl Acid Res. 2003;31:833-43), and the pro-inflammatory ICAM-1 gene (Besch et al, J Biol Chem, 2002;277:32473-79). In addition, Vuyisich and Beal have recently shown that sequence specific TFOs can bind to dsRNA, inhibiting activity of dsRNA-dependent enzymes such as RNA-dependent kinases (Vuyisich and Beal, Nuc. Acids Res 2000;28:2369-74).

Additionally, TFOs designed according to the abovementioned principles can induce directed mutagenesis capable of effecting DNA repair, thus providing both downregulation and upregulation of expression of endogenous genes (Seidman and Glazer, J Clin Invest 2003;112:487-94). Detailed description of the design, synthesis and administration of effective TFOs can be found in U.S. Patent Application Nos. 2003 017068 and 2003 0096980 to Froehler et al, and 2002 0128218 and 2002 0123476 to Emanuele et al, and U.S. Pat. No. 5,721,138 to Lawn.

Another agent capable of downregulating NOTCH pathway component would be any molecule which binds to and/or cleaves the component. Such molecules can be NOTCH pathway component antagonists, or NOTCH pathway component inhibitory peptides.

It will be appreciated that a non-functional analogue of at least a catalytic or binding portion of NOTCH pathway component can be also used as an agent of the present invention.

Another agent which can be used along with the present invention to downregulate NOTCH pathway component is a molecule which prevents NOTCH receptor activation or substrate binding.

Polypeptide agents (e.g. antibodies) for up-regulating beta cell differentiation may be provided to the adult islet beta cells per se. Polynucleotide agents for up-regulating beta cell differentiation are typically administered to the adult islet beta cells as part of an expression construct. In this case, the polynucleotide agent is ligated in a nucleic acid construct under the control of a cis-acting regulatory element (e.g. promoter) capable of directing an expression of the agent capable of downregulating the NOTCH pathway component in the adult islet beta cells in a constitutive or inducible manner.

The nucleic acid construct may be introduced into the expanded cells of the present invention using an appropriate gene delivery vehicle/method (transfection, transduction, etc.) and an appropriate expression system. Examples of suitable constructs include, but are not limited to, pcDNA3, pcDNA3.1 (+/−), pGL3, PzeoSV2 (+/−), pDisplay, pEF/myc/cyto, pCMV/myc/cyto each of which is commercially available from Invitrogen Co. (www.invitrogen.com). Lipid-based systems may be used for the delivery of these constructs into the expanded adult islet beta cells of the present invention. Useful lipids for lipid-mediated transfer of the gene are, for example, DOTMA, DOPE, and DC-Chol [Tonkinson et al., Cancer Investigation, 14(1): 54-65 (1996)]. Recently, it has been shown that Chitosan can be used to deliver nucleic acids to the intestine cells (Chen J. (2004) World J Gastroenterol 10(1):112-116). Other non-lipid based vectors that can be used according to this aspect of the present invention include but are not limited to polylysine and dendrimers.

The expression construct may also be a virus. Examples of viral constructs include but are not limited to adenoviral vectors, retroviral vectors, vaccinia viral vectors, adeno-associated viral vectors, polyoma viral vectors, alphaviral vectors, rhabdoviral vectors, lenti viral vectors and herpesviral vectors.

A viral construct such as a retroviral construct includes at least one transcriptional promoter/enhancer or locus-defining element(s), or other elements that control gene expression by other means such as alternate splicing, nuclear RNA export, or post-transcriptional modification of messenger. Such vector constructs also include a packaging signal, long terminal repeats (LTRs) or portions thereof, and positive and negative strand primer binding sites appropriate to the virus used, unless it is already present in the viral construct. In addition, such a construct typically includes a signal sequence for secretion of the peptide from a host cell in which it is placed. Preferably, the signal sequence for this purpose is a mammalian signal sequence or the signal sequence of the peptide variants of the present invention. Optionally, the construct may also include a signal that directs polyadenylation, as well as one or more restriction site and a translation termination sequence. By way of example, such constructs will typically include a 5′ LTR, a tRNA binding site, a packaging signal, an origin of second-strand DNA synthesis, and a 3′ LTR or a portion thereof.

Preferably the viral dose for infection is at least 10³, 10⁴, 10⁵, 10⁶, 10⁷, 10⁸, 10⁹, 10¹⁰, 10¹¹, 10¹², 10¹³, 10¹⁴, 10¹⁵ or higher pfu or viral particles.

Another agent capable of down-regulating a component of the NOTCH pathway is a gamma secretase inhibitor. Gamma secretase is required to cleave the NOTCH receptor and generate the active form which then translocates into the nucleus. Exemplary gamma secretase inhibitors contemplated by the present invention include, but are not limited to III-31-C; N-[N-(3,5-difluorophenacetyl)-L-alanyl]S-phenylglycine t-butyl ester) (DAPT); compound E; D-helical peptide 294; isocoumarins; BOC-Lys(Cbz)Ile-Leu-epoxide; and (Z-LL)₂-ketone and (S, S)-2-[2-(3,5-difluorophenyl)acetylamino]-N-(5-methyl-6-oxo-6,7-dihydro-5H-dibenzo [b, d]azepin-7-yl)propionamide (DBZ).

The agents capable of down-regulating a component of the NOTCH pathway are typically provided in a quantity that is sufficient to increase insulin content in the adult islet beta cells. The phrase “insulin content” refers to the amount of mature insulin inside an adult beta cell. Measurement of insulin content is well known in the art. An exemplary method is extraction of cellular insulin with 3 M acetic acid. The amount of mature insulin extracted from the adult islet beta cells may be determined using an ELISA kit commercially available from Mercodia, Uppsala, Sweden.

Any medium may be used to incubate the expanded adult islet beta cells in the presence of the agent capable of down-regulating a component of the NOTCH pathway. According to one embodiment, the medium is CMRL-1066.

The adult islet beta cells of the present invention may be further modified (e.g. genetic modification) during or following the redifferentiation stage to express a pharmaceutical agent such as a therapeutic agent, a telomerase gene, an agent that reduces immune mediated rejection or a marker gene. It is contemplated that therapeutic agents such as antimetabolites (e.g., purine analogs, pyrimidine analogs), enzyme inhibitors and peptidomimetics may be generally useful in the present invention. An example of a gene that may reduce immune mediated rejection is the uteroglobin gene. Uteroglobin is a protein expressed during pregnancy that confers immunologic tolerance and prevents inflammatory reactions. Methods of genetically modifying the adult islet beta cells of the present invention are described hereinabove.

Since the redifferentiated adult islet pancreatic cells of the present invention store and secrete insulin, they may be used for treating a disease which is associated with insulin deficiency such as diabetes.

Thus, according to another aspect of the present invention there is provided a method of treating diabetes in a subject, the method comprising transplanting a therapeutically effective amount of the population of re-differentiated, expanded adult islet beta cells into the subject.

As used herein “diabetes” refers to a disease resulting either from an absolute deficiency of insulin (type 1 diabetes) due to a defect in the biosynthesis or production of insulin, or a relative deficiency of insulin in the presence of insulin resistance (type 2 diabetes), i.e., impaired insulin action, in an organism. The diabetic patient thus has absolute or relative insulin deficiency, and displays, among other symptoms and signs, elevated blood glucose concentration, presence of glucose in the urine and excessive discharge of urine.

The phrase “treating” refers to inhibiting or arresting the development of a disease, disorder or condition and/or causing the reduction, remission, or regression of a disease, disorder or condition in an individual suffering from, or diagnosed with, the disease, disorder or condition. Those of skill in the art will be aware of various methodologies and assays which can be used to assess the development of a disease, disorder or condition, and similarly, various methodologies and assays which can be used to assess the reduction, remission or regression of a disease, disorder or condition.

As used herein, “transplanting” refers to providing the redifferentiated adult islet beta cells of the present invention, using any suitable route. Typically, beta cell therapy is effected by injection using a catheter into the portal vein of the liver, although other methods of administration are envisaged.

As mentioned hereinabove, the adult islet beta cells of the present invention can be derived from either autologous sources or from allogeneic sources such as human cadavers or donors. Since non-autologous cells are likely to induce an immune reaction when administered to the body several approaches have been developed to reduce the likelihood of rejection of non-autologous cells. These include either suppressing the recipient immune system or encapsulating the non-autologous cells in immunoisolating, semipermeable membranes before transplantation.

Encapsulation techniques are generally classified as microencapsulation, involving small spherical vehicles and macroencapsulation, involving larger flat-sheet and hollow-fiber membranes (Uludag, H. et al. Technology of mammalian cell encapsulation. Adv Drug Deliv Rev. 2000; 42: 29-64).

Methods of preparing microcapsules are known in the arts and include for example those disclosed by Lu M Z, et al., Cell encapsulation with alginate and alpha-phenoxycinnamylidene-acetylated poly(allylamine). Biotechnol Bioeng. 2000, 70: 479-83, Chang T M and Prakash S. Procedures for microencapsulation of enzymes, cells and genetically engineered microorganisms. Mol Biotechnol. 2001, 17: 249-60, and Lu M Z, et al., A novel cell encapsulation method using photosensitive poly(allylamine alpha-cyanocinnamylideneacetate). J Microencapsul. 2000, 17: 245-51.

For example, microcapsules are prepared by complexing modified collagen with a ter-polymer shell of 2-hydroxyethyl methylacrylate (HEMA), methacrylic acid (MAA) and methyl methacrylate (MMA), resulting in a capsule thickness of 2-5 μm. Such microcapsules can be further encapsulated with additional 2-5 μm ter-polymer shells in order to impart a negatively charged smooth surface and to minimize plasma protein absorption (Chia, S. M. et al. Multi-layered microcapsules for cell encapsulation Biomaterials. 2002 23: 849-56).

Other microcapsules are based on alginate, a marine polysaccharide (Sambanis, A. Encapsulated islets in diabetes treatment. Diabetes Thechnol. Ther. 2003, 5: 665-8) or its derivatives. For example, microcapsules can be prepared by the polyelectrolyte complexation between the polyanions sodium alginate and sodium cellulose sulphate with the polycation poly(methylene-co-guanidine) hydrochloride in the presence of calcium chloride.

It will be appreciated that cell encapsulation is improved when smaller capsules are used. Thus, the quality control, mechanical stability, diffusion properties, and in vitro activities of encapsulated cells improved when the capsule size was reduced from 1 mm to 400 μm (Canaple L. et al., Improving cell encapsulation through size control. J Biomater Sci Polym Ed. 2002;13:783-96). Moreover, nanoporous biocapsules with well-controlled pore size as small as 7 nm, tailored surface chemistries and precise microarchitectures were found to successfully immunoisolate microenvironments for cells (Williams D. Small is beautiful: microparticle and nanoparticle technology in medical devices. Med Device Technol. 1999, 10: 6-9; Desai, T. A. Microfabrication technology for pancreatic cell encapsulation. Expert Opin Biol Ther. 2002, 2: 633-46).

Examples of immunosuppressive agents include, but are not limited to, methotrexate, cyclophosphamide, cyclosporine, cyclosporin A, chloroquine, hydroxychloroquine, sulfasalazine (sulphasalazopyrine), gold salts, D-penicillamine, leflunomide, azathioprine, anakinra, infliximab (REMICADE.sup.R), etanercept, TNF.alpha. blockers, a biological agent that targets an inflammatory cytokine, and Non-Steroidal Anti-Inflammatory Drug (NSAIDs). Examples of NSAIDs include, but are not limited to acetyl salicylic acid, choline magnesium salicylate, diflunisal, magnesium salicylate, salsalate, sodium salicylate, diclofenac, etodolac, fenoprofen, flurbiprofen, indomethacin, ketoprofen, ketorolac, meclofenamate, naproxen, nabumetone, phenylbutazone, piroxicam, sulindac, tolmetin, acetaminophen, ibuprofen, Cox-2 inhibitors and tramadol.

It will be appreciated that the agent capable of downregulating a component of the NOTCH pathway may be administered directly to a subject (for example in a nucleic acid carrier, such as a liposome) in order to increase insulin production in the pancreas thereof—i.e. in vivo treatment.

The redifferentiated adult islet beta cells of the present invention may be transplanted to a subject per se, or in a pharmaceutical composition where they are mixed with suitable carriers or excipients. Similarly, the agent of the present invention may be administered to a subject per se, or in a pharmaceutical composition.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the adult islet beta cells of the present invention accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, inrtaperitoneal, intranasal, or intraocular injections. Alternately, one may administer the pharmaceutical composition in a local rather than systemic manner, for example, via injection of the pharmaceutical composition directly into a tissue region of a patient.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of active ingredients (insulin producing cells) effective to prevent, alleviate or ameliorate symptoms of a disorder (e.g., diabetes) or prolong the survival of the subject being treated.

Determination of a therapeutically effective amount is well within the capability of those skilled in the art, especially in light of the detailed disclosure provided herein. For any preparation used in the methods of the invention, the therapeutically effective amount or dose can be estimated from animal models (e.g. STZ diabetic mice) to achieve a desired concentration or titer. Such information can be used to more accurately determine useful doses in humans.

Toxicity and therapeutic efficacy of the active ingredients described herein can be determined by standard pharmaceutical procedures in experimental animals. The data obtained from these animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide cell numbers sufficient to induce normoglycemia (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as if further detailed above.

As mentioned herein above, dedifferentiated beta cells may be purified prior to redifferentiation and following expansion.

Thus, according to another aspect of the present invention there is provided a method of purifying a population of dedifferentiated B cells, the method comprising:

(a) permanently tagging primary B cells of cultured human islets, wherein the tagging is irrespective of a subsequent differentiation status of the B cells, to generate a population of permanently tagged B cells;

(b) culturing the permanently tagged B cells under conditions sufficient to allow dedifferentiation of the tagged B cells to generate a population of dedifferentiated tagged B cells; and

(c) isolating the population of dedifferentiated tagged B cells, thereby purifying the population of dedifferentiated B cells.

As used herein, the phrase “purifying a population of dedifferentiated B cells” refers to isolating dedifferentiated B cells such that 80% or more of the resultant cell population comprises dedifferentiated B cells. According to one embodiment, 90% or more of the resultant cell population comprises dedifferentiated B cells. According to another embodiment, 95% or more of the resultant cell population comprises dedifferentiated B cells. According to another embodiment, 99% or more of the resultant cell population comprises dedifferentiated B cells.

The phrase “permanently tagging” refers to incorporating a detectable moiety into, or on the surface of, the cells such that the detectable moiety remains in/on the cell irrespective of the differentiation status of the cell.

According to this aspect of the present invention, the tagging is effected prior to the process of dedifferentiation, whilst the B cell still expresses B cell markers (e.g. insulin). Typically, the tagging is effected no more than five days following culturing, and more preferably no more than three days following culturing.

An exemplary method for permanently tagging cells is described in Example 3 herein below.

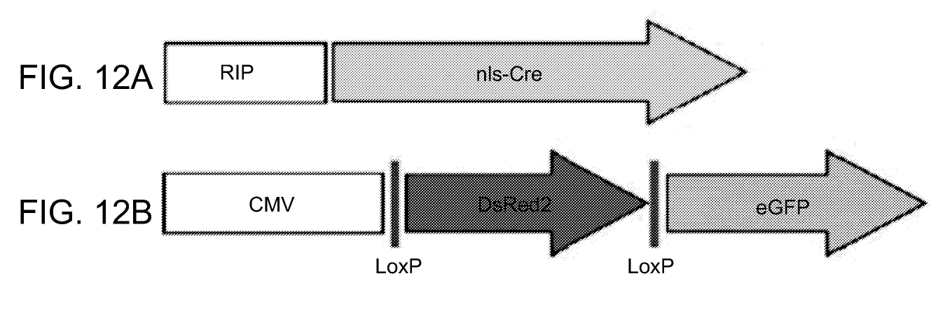

In brief, the B cells are transfected with two expression constructs—see FIGS. 12A-B. The first expression construct comprises a polynucleotide encoding a Cre recombinase polypeptide operatively linked to a B cell specific promoter.

Examples of B cell specific promoters include, but are not limited to an insulin promoter or a Pdx1 promoter.

According to one embodiment of this aspect of the present invention, the first expression construct comprises a polynucleotide comprising a nucleic acid sequence as set forth in SEQ ID NO: 11.

The second expression construct comprises a first polynucleotide encoding a first detectable moiety operatively linked to a constitutive promoter, the first polynucleotide being flanked by LoxP polynucleotides. The second expression construct further comprises a second polynucleotide encoding a second detectable moiety, the second polynucleotide being positioned 3′ to the first polynucleotide.

According to another embodiment of this aspect of the present invention, the second expression construct comprises a polynucleotide comprising a nucleic acid sequence as set forth in SEQ ID NO: 12.

The expression constructs of the present invention may also include additional sequences which render it suitable for replication and integration in eukaryotes (e.g., shuttle vectors). Typical cloning vectors contain transcription and translation initiation sequences (e.g., promoters, enhances) and transcription and translation terminators (e.g., polyadenylation signals). The expression constructs of the present invention can further include an enhancer, which can be adjacent or distant to the promoter sequence and can function in up regulating the transcription therefrom.

Enhancer elements can stimulate transcription up to 1,000-fold from linked homologous or heterologous promoters. Enhancers are active when placed downstream or upstream from the transcription initiation site. Many enhancer elements derived from viruses have a broad host range and are active in a variety of tissues. For example, the SV40 early gene enhancer is suitable for many cell types. Other enhancer/promoter combinations that are suitable for the present invention include those derived from polyoma virus or human or murine cytomegalovirus (CMV) and the long tandem repeats (LTRs) from various retroviruses, such as murine leukemia virus, murine or Rous sarcoma virus, and HIV. See Gluzman, Y. and Shenk, T., eds. (1983). Enhancers and Eukaryotic Gene Expression, Cold Spring Harbor Press, Cold Spring Harbor, N.Y., which is incorporated herein by reference.

Polyadenylation sequences can also be added to the expression constructs of the present invention in order to increase the efficiency of expression of the detectable moiety. Two distinct sequence elements are required for accurate and efficient polyadenylation: GU- or U-rich sequences located downstream from the polyadenylation site and a highly conserved sequence of six nucleotides, namely AAUAAA, located 11-30 nucleotides upstream of the site. Termination and polyadenylation signals suitable for the present invention include those derived from SV40.

In addition to the embodiments already described, the expression constructs of the present invention may typically contain other specialized elements intended to increase the level of expression of cloned nucleic acids or to facilitate the identification of cells that carry the recombinant DNA. For example, a number of animal viruses contain DNA sequences that promote extra-chromosomal replication of the viral genome in permissive cell types. Plasmids bearing these viral replicons are replicated episomally as long as the appropriate factors are provided by genes either carried on the plasmid or with the genome of the host cell.

The expression constructs of the present invention may or may not include a eukaryotic replicon. If a eukaryotic replicon is present, the vector is capable of amplification in eukaryotic cells using the appropriate selectable marker. If the construct does not comprise a eukaryotic replicon, no episomal amplification is possible. Instead, the recombinant DNA integrates into the genome of the engineered cell, where the promoter directs expression of the desired nucleic acid.

Examples of mammalian expression vectors include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, and pNMT81, which are available from Invitrogen, pCI which is available from Promega, pMbac, pPbac, pBK-RSV and pBK-CMV, which are available from Strategene, pTRES which is available from Clontech, and their derivatives.

Expression vectors containing regulatory elements from eukaryotic viruses such as retroviruses can be also used. SV40 vectors include pSVT7 and pMT2, for instance. Vectors derived from bovine papilloma virus include pBV-1MTHA, and vectors derived from Epstein-Ban virus include pHEBO and p2O5. Other exemplary vectors include pMSG, pAV009/A⁺, pMTO10/A⁺, pMAMneo-5 and baculovirus pDSVE.

Retroviral vectors represent a class of vectors particularly suitable for use with the present invention. Defective retroviruses are routinely used in transfer of genes into mammalian cells (for a review, see Miller, A. D. (1990). Blood 76, 271). A recombinant retrovirus comprising the polynucleotides of the present invention can be constructed using well-known molecular techniques. Portions of the retroviral genome can be removed to render the retrovirus replication machinery defective, and the replication-deficient retrovirus can then packaged into virions, which can be used to infect target cells through the use of a helper virus while employing standard techniques. Protocols for producing recombinant retroviruses and for infecting cells with viruses in vitro or in vivo can be found in, for example, Ausubel et al. (1994) Current Protocols in Molecular Biology (Greene Publishing Associates, Inc. & John Wiley & Sons, Inc.). Retroviruses have been used to introduce a variety of genes into many different cell types, including neuronal cells, epithelial cells, endothelial cells, lymphocytes, myoblasts, hepatocytes, and bone marrow cells.

According to one embodiment, a lentiviral vector, a type of retroviral vector, is used according to the present teachings. Lentiviral vectors are widely used as vectors due to their ability to integrate into the genome of non-dividing as well as dividing cells. The viral genome, in the form of RNA, is reverse-transcribed when the virus enters the cell to produce DNA, which is then inserted into the genome at a random position by the viral integrase enzyme. The vector (a provirus) remains in the genome and is passed on to the progeny of the cell when it divides. For safety reasons, lentiviral vectors never carry the genes required for their replication. To produce a lentivirus, several plasmids are transfected into a so-called packaging cell line, commonly HEK 293. One or more plasmids, generally referred to as packaging plasmids, encode the virion proteins, such as the capsid and the reverse transcriptase. Another plasmid contains the genetic material to be delivered by the vector. It is transcribed to produce the single-stranded RNA viral genome and is marked by the presence of the ψ (psi) sequence. This sequence is used to package the genome into the virion.

A specific example of a suitable lentiviral vector for introducing and expressing the polynucleotide sequences of the present invention in B cells is the lentivirus pLKO.1 vector.

Another suitable expression vector that may be used according to this aspect of the present invention is the adenovirus vector. The adenovirus is an extensively studied and routinely used gene transfer vector. Key advantages of an adenovirus vector include relatively high transduction efficiency of dividing and quiescent cells, natural tropism to a wide range of epithelial tissues, and easy production of high titers (Russel, W. C. (2000) J Gen Virol 81, 57-63). The adenovirus DNA is transported to the nucleus, but does not integrate thereinto. Thus the risk of mutagenesis with adenoviral vectors is minimized, while short-term expression is particularly suitable for treating cancer cells. Adenoviral vectors used in experimental cancer treatments are described by Seth et al. (1999). “Adenoviral vectors for cancer gene therapy,” pp. 103-120, P. Seth, ed., Adenoviruses: Basic Biology to Gene Therapy, Landes, Austin, Tex.).

A suitable viral expression vector may also be a chimeric adenovirus/retrovirus vector combining retroviral and adenoviral components. Such vectors may be more efficient than traditional expression vectors for transducing tumor cells (Pan et al. (2002). Cancer Letts 184, 179-188).

Various methods can be used to introduce the expression vectors of the present invention into human embryonic stem cells. Such methods are generally described in, for instance: Sambrook, J. and Russell, D. W. (1989, 1992, 2001), Molecular Cloning: A Laboratory Manual, Cold Springs Harbor Laboratory, New York; Ausubel, R. M. et al., eds. (1994, 1989). Current Protocols in Molecular Biology, John Wiley and Sons, Baltimore, Md. (1989); Chang, P. L., ed. (1995). Somatic Gene Therapy, CRC Press, Boca Raton, Fla.; Vega, M. A. (1995). Gene Targeting, CRC Press, Boca Raton, Fla.; Rodriguez, R. L. and Denhardt, D. H. (1987). Vectors: A Survey of Molecular Cloning Vectors and Their Uses, Butterworth-Heinemann, Boston, Mass; and Gilboa, E. et al. (1986). Transfer and expression of cloned genes using retro-viral vectors. Biotechniques 4(6), 504-512; and include, for example, stable or transient transfection, lipofection, electroporation, and infection with recombinant viral vectors. In addition, see U.S. Pat. Nos. 5,464,764 and 5,487,992 for positive-negative selection methods.

Introduction of the expression constructs of the present invention into beta cells by viral infection offers several advantages over other methods such as lipofection and electroporation offering higher efficiency of transformation and propagation.

It will be appreciated that the tag (i.e. detectable moiety) may be any polypeptide which can be detected in a B cell throughout the course of its dedifferentiation, which itself does not influence B cell viability or dedifferentiation.

According to one embodiment, the tag is a light emitting protein.

Examples of tags which may be detected in B cells include, but are not limited to, light emitting protein genes such as green fluorescent proteins including EGFP (Enhanced Green Fluorescent Protein) and GFP (Green Fluorescent Protein), blue fluorescent protein (EBFP, EBFP2, Azurite, mKa1ama1), cyan fluorescent protein (ECFP, Cerulean, CyPet) and yellow fluorescent protein derivatives (YFP, Citrine, Venus, YPet) and LacZ gene.

As used herein, the phrase “primary B cells of cultured human islets” refers to B cells (that express insulin and other B cell markers) that have been removed from their in vivo environment and cultured directly without transformation.

As mentioned, following tagging, the cells are cultured under conditions which allow dedifferentiation.

To enable dedifferentiation of primary B cells, the cells are allowed to divide in culture medium (for example CMRL) for at least one week and preferably no more than 16 weeks to prevent B cell apoptosis.

Following culturing, the cells are isolated. Exemplary methods of isolating tagged cells include, but are not limited to manual dissection (microdis section) using a microscope capable of detecting the tag (e.g. fluorescent microscope) and sorting using a FACS sorter.

Purified populations of dedifferentiated B cells may be used for a variety of purposes. According to one embodiment, they are used for screening candidate agents which affect proliferation and/or redifferentiation of dedifferentiated B cells. Exemplary candidate agents include, but are not limited to small molecules, polypeptide agents and polynucleotide agents (e.g. siRNAs).

### Examples

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorpotaed by reference as if fully set forth herein. Other general references are provided throughout this document. The procedures therein are believed to be well known in the art and are provided for the convenience of the reader. All the information contained therein is incorporated herein by reference.

### Example 1

### HES1 is Involved in Adaptation of Adult Human Beta Cells to Proliferation In Vitro

Materials and Methods

Islet cell culture: Islets were received 2-3 days following isolation. Islets from individual donors were dissociated into single cells and cultured in CMRL 1066 medium containing 5.6 mM glucose and supplemented with 10% fetal bovine serum (FBS), 100 U/ml penicillin, 100 μg/ml streptomycin, 100 μg/ml gentamycin, and 5 μg/ml amphotericine B as described [Ouziel Yahalom et al., Biochem Biophys Res Commun 341:291-298, 2006]. The cultures were fed twice a week and split 1:2 once a week.

HES1 inhibition and lineage tracing: HES1 shRNA (TGGCCAGTTTGCTTTCCTCAT)—SEQ ID NO: 7 and a non-target shRNA, cloned in pLKO.1 lentiviral vector, were obtained from the RNAi Consortium (Sigma-Aldrich). Virus was produced in 293T cells following cotransfection with the pCMVdR8.91 and pMD2.G packaging plasmids. The culture medium was harvested 48 hours later. Islet cells cultured for 1-2 days were washed with PBS and infected at MOI 2.5:1 in CMRL 1066 medium containing 8 μg/ml polybrene overnight. The medium was then replaced with regular culture medium. Four days after infection, the cells were selected for puromycin resistance (1 μg/ml) for 3 days. Two weeks after infection the cells were harvested for further analysis. Lineage tracing was performed using the RIP-Cre and a pTrip CMV-loxP-DsRed2-loxP-eGFP viruses as described herein below. Briefly, islet cells cultured for 1-2 days were infected with a 1:1:1 mixture of the 3 viruses (shRNA+RIP-Cre+pTrip CMV-loxP-DsRed2-loxP-eGFP) at a total MOI 4:1. Selection and further analysis were carried out as above.

RNA analyses: Total RNA was extracted using High Pure RNA isolation kit (Roche). cDNA was synthesized using SuperScript III (Invitrogen). qPCR was performed using a Prism 7300 ABI Real Time PCR System (Applied Biosystems). The Assay-On-Demand (Applied Biosystems) TaqMan fluorogenic probes that were used in this study are listed in Table 1, herein below. Relative quantitative analysis was performed according to the comparative CT method by using the arithmetic formula 2̂^{-(ΔΔct)}. The cDNA levels were normalized to human ribosomal protein P0 (RPLP0) cDNA.

TABLE 1
TaqMan fluorogenic probes

| Gene | Probe |
|---|---|
| DLL1 | Hs00194509_m1 |
| HES1 | Hs00172878_m1 |
| Insulin | Hs00355773_m1 |
| JAG2 | Hs00171432_m1 |
| NEUROD1 | Hs00159598_m1 |
| NOTCH1 | Hs00413187_m1 |
| NOTCH2 | Hs01050706_m1 |
| NOTCH3 | Hs01128547_m1 |
| NOTCH4 | Hs00965897_m1 |
| p57 | Hs00175938_m1 |
| PDX1 | Hs00426216_m1 |
| RPLP0 | Hs99999902_m1 |

Immunofluorescence: Cells were plated in 24-well plates on sterilized coverslips and fixed in 4% paraformaldehyde. Cells were permeabilized with 0.25% NP40 for 10 minutes and blocked for 10 minutes at room temperature in 1% bovine serum albumin, 10% FBS, and 0.2% saponin. Cells were then incubated with the following primary antibodies diluted in blocking solution, overnight at 4° C.: mouse-anti-insulin (Sigma-Aldrich, 1:1000); Rabbit-anti-p57 (Santa Cruz,1:500); rabbit-anti-HES1 (Chimicon, 1:1000); mouse-anti-Ki67 (Zymed, 1:200); rabbit-anti-Ki67 (Zymed, 1:50); mouse-anti-BrdU (1:20); rabbit-anti-NICD (Cell Signalling, 1:10); mouse-anti-GFP (Chemicon, 1:500); and rabbit-anti-GFP (Invitrogen, 1:1000). The bound antibody was visualized with a fluorescent secondary antibody: anti-mouse- or anti-rabbit-AMCA (Jackson, 1:200); -Cy3 (Biomeda, 1:200); and -Alexa Fluor 488 (Molecular Probes, 1:200), under a Zeiss confocal microscope. The specificity of the primary antibodies was demonstrated using human fibroblast cells (data not shown). Nuclei were visualized by staining with DAPI (Roche) for 5 min at room temperature. BrdU staining was performed following a 24-hour labeling period as previously described (Berkovich and Efrat, Diabetes 50:2260-2267, 2001).

Immunoblotting: Total cellular protein was extracted in 0.5% NP40 containing a protease inhibitor cocktail (Roche). Protein concentration was determined by the BCA method (Pierce, Rockford, Ill.). 70 μg protein were separated on 12% sodium dodecyl sulphate-polyacrylamide gels and electroblotted onto PDF membranes. The membranes were incubated with rabbit-anti-HES1 (1:1000) or rabbit-anti-PARP (Cell Signaling, 1:1000). Loading was monitored using goat-anti-beta-actin (Santa Cruz, 1:1000). The bound antibody was visualized with the appropriate horseradish peroxidase-conjugated anti-IgG (Jackson) and SuperSignal West Pico Chemiluminescent Substrate (Pierce). Cells treated with 1 μm staurosporine for 6 hours were used as positive control for the PARP blot.

Statistical Analysis: Significance was determined using Student's t-test.

Results

Up-regulation of HES1 in cultured beta cells: Human islets were isolated from 9 donors, 6 males and 3 females, aged 38-60 (mean age 50±8), with a purity ranging between 65-85% (mean 78±6%). Islets from each donor were dissociated and expanded in culture as described [Ouziel Yahalom et al., Biochem Biophys Res Commun 341:291-298, 2006]. Quantitative RT-PCR (qPCR) analyses of RNA extracted from these cells during the first 2 weeks of culture revealed a rapid dedifferentiation, as previously reported [Ouziel Yahalom et al., Biochem Biophys Res Commun 341:291-298, 2006], which was manifested in a drastic decrease in insulin mRNA levels, averaging 166-fold (p<1.2×10⁻¹⁸) (FIG. 1A). Concomitant with this decrease, an increase in HES1 mRNA was observed in cells from all donors, averaging 5.3-fold (p<0.0004) within the first 2 week of culture (FIG. 1B). A similar increase was noted in HES1 protein levels (FIG. 1C). At both RNA and protein levels the wave of HES1 upregulation peaked within the first 2 weeks of culture and was downregulated thereafter. Immunostaining could not detect significant HES1 expression in nuclei of cells with intense insulin staining (FIG. 1D). In contrast, HES1 was clearly detected in insulin-negative cells. To monitor HES1 expression in dedifferentiated cells derived from beta cells, beta cells in freshly-isolated islets were heritably labeled using a cell-lineage tracing approach. The labeling approach is based on cell infection with a mixture of 2 lentivirus vectors, one expressing Cre recombinase under the insulin promoter (RIP-Cre), and the other a reporter cassette in which the CMV promoter is separated from an eGFP gene by a loxP-flanked stop region. Removal of the stop region in beta cells infected by both viruses activates eGFP expression specifically in these cells, thereby allowing continuous tracking of beta-cell fate after insulin expression is lost. Residual insulin expression in beta cells during the initial days in culture provides a sufficient window of time for RIP-Cre expression and eGFP activation. Analysis of the cells expanded in culture following labeling revealed HES1 staining in cells that lost insulin expression but maintained eGFP expression, demonstrating that they were derived from beta cells (FIGS. 1D-E).

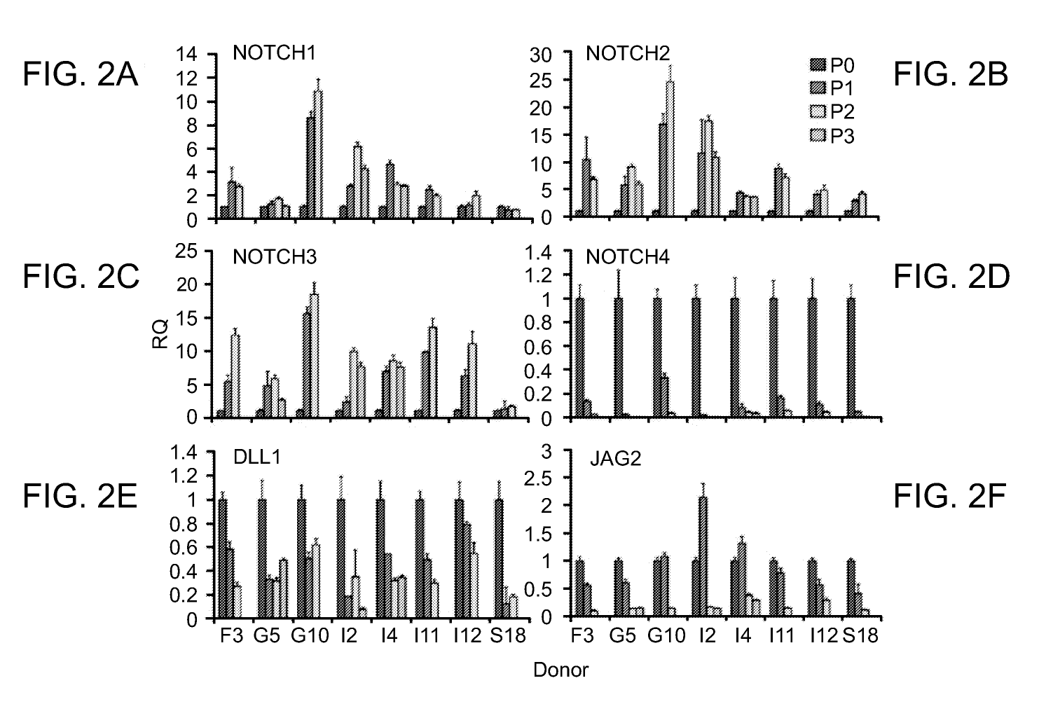

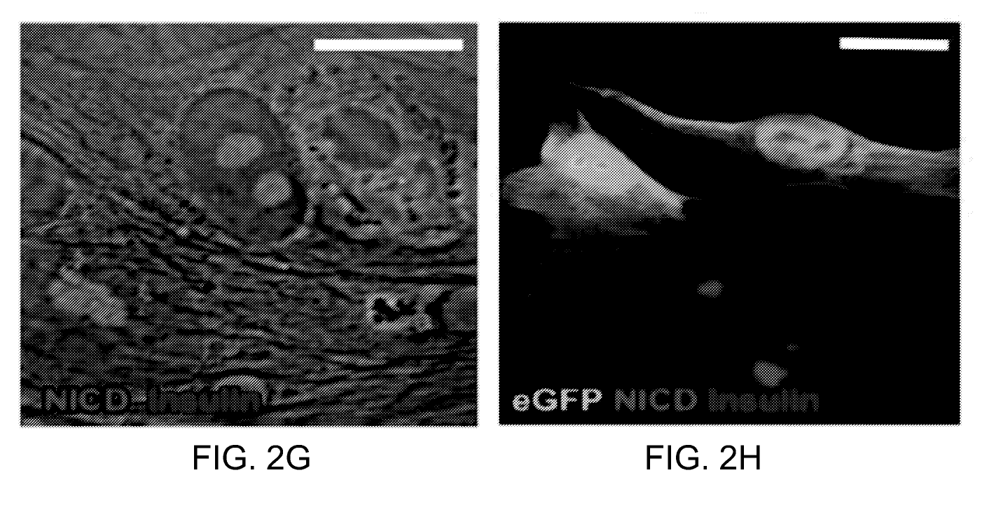

Changes in expression of components of the NOTCH pathway in cultured beta cells. qPCR analyses revealed changes in levels of transcripts encoding the 4 members of the NOTCH family. NOTCH1 transcripts were upregulated on average by 3.9-fold within the first 2 week of culture (p<0.03) (FIG. 2A). NOTCH2 and NOTCH3 were significantly upregulated on average by 10.3-fold (p<0.009) and 10.1-fold (p<0.001), respectively, within the first 2 week of culture (FIGS. 2B-C). Overall, the activation of NOTCH1-3 paralleled that of HES1. In contrast, NOTCH4 was drastically downregulated on average 50-fold (p<1.5×10⁻¹³) from its level in primary islets (FIG. 2D). As with HES1 upregulation, NOTCH1-3 upregulation peaked within the first 2 weeks of culture and was downregulated thereafter. Transcripts encoding presenilin 1, a protein required for generation of NICD, and RBPJK, a protein that participates in the NICD nuclear complex, were not significantly changed in the cultured cells (data not shown). In contrast, transcripts for NOTCH ligands were downregulated during the initial weeks of culture (FIGS. 2E-F). DELTA1 was downregulated on average 3.1-fold (p<2.7×10⁻⁶) within the first 2 week of culture. JAG1 was not significantly changed (data not shown). JAG2 was downregulated on average 5.5-fold (p<7.9×10⁻⁸) within the first 2 week of culture. The increased activity of the NOTCH pathway was manifested by appearance of NICD in cell nuclei, as revealed by immunostaining (FIGS. 2G-H). Similar to the pattern of HES1 immunostaining, staining for NICD could not be detected in cells intensely stained for insulin. NICD staining was detected in lineage-labeled insulin-negative cells identified as originating from beta cells by eGFP expression (FIGS. 2G-H).

Changes in expression of cell cycle inhibitors: To evaluate the consequences of increased HES1 expression in the cultured islet cells, changes in transcripts of genes encoding cyclin kinase inhibitors, (which are among the main targets of repression by HES1), were analyzed. Transcripts encoding p57, which is thought to be the main cell cycle inhibitor in human beta cells, were downregulated on average 2.8-fold (p<0.0003) within the first 2 weeks of culture (FIG. 3A). This finding was supported by immunostaining for p57, which showed its presence in lineage-labeled insulin-positive, eGFP⁺ cells, and its absence in insulin-negative, eGFP⁺ cells (FIGS. 3B-D). In contrast to p57, transcripts for p21 were upregulated in cells from all donors, and those for p27 varied considerably among donors (data not shown). The downregulation of p57 transcripts and protein correlated with cell entrance into the cell cycle, as manifested by Ki67 staining in p57-negative, eGFP⁺ cells (FIGS. 3E-G).

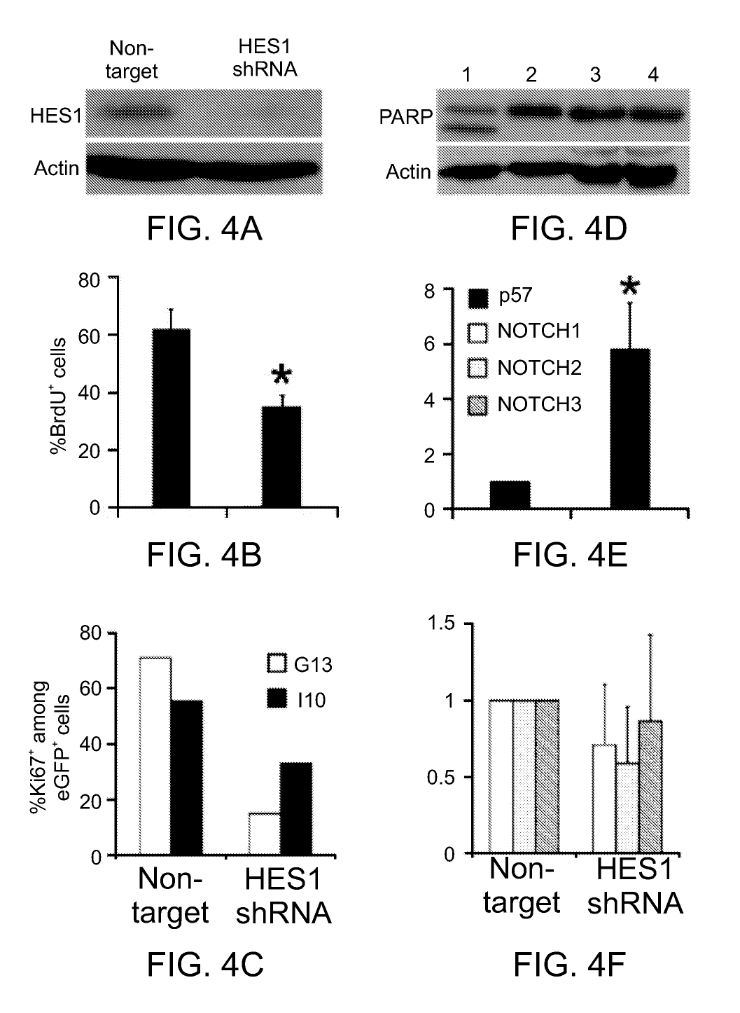

Inhibition of HES1 expression prevents induction of beta-cell replication: To further correlate the induction of beta-cell replication with HES1 upregulation, HES1 induction during the initial weeks of culture was inhibited using shRNA. Following screening of 4 HES1 shRNA sequences for activity in 293T cells, one of the four was selected as most efficient (SEQ ID NO: 7) based on reduction in HES1 protein levels, as analyzed by immunoblotting. Isolated human islets were dissociated, and the cells were infected with a lentivirus encoding HES1 shRNA before culture under standard conditions. Selection for drug resistance allowed elimination of uninfected cells. Cells infected with a non-target shRNA lentivirus and selected under similar conditions served as control. As seen in FIG. 4A, cell infection with the HES1 shRNA virus resulted in up to 6× lower HES1 protein levels, compared with cells infected with the control virus. The lower HES1 levels were associated with a diminished cell proliferation, compared with cells infected with the control vector, as judged by staining for BrdU incorporation (FIG. 4B). In addition, staining for Ki67 in eGFP⁺ cells demonstrated a lower replication rate among cells derived from beta cells (FIG. 4C). The reduced replication in cells infected with the HES1 shRNA virus did not correlate with an increase in cell apoptosis, as judged by immunoblotting analysis for cleaved poly(ADP-ribose) polymerase (PARP) (FIG. 4D). The reduced proliferation correlated with a 5.7-fold (p<0.04) higher level of p57 transcripts, compared with those in cells infected with the control virus (FIG. 4E). The reduced HES1 levels did not affect the levels of NOTCH transcripts, which is consistent with the position of HES1 downstream of NOTCH in the pathway (FIG. 4F).

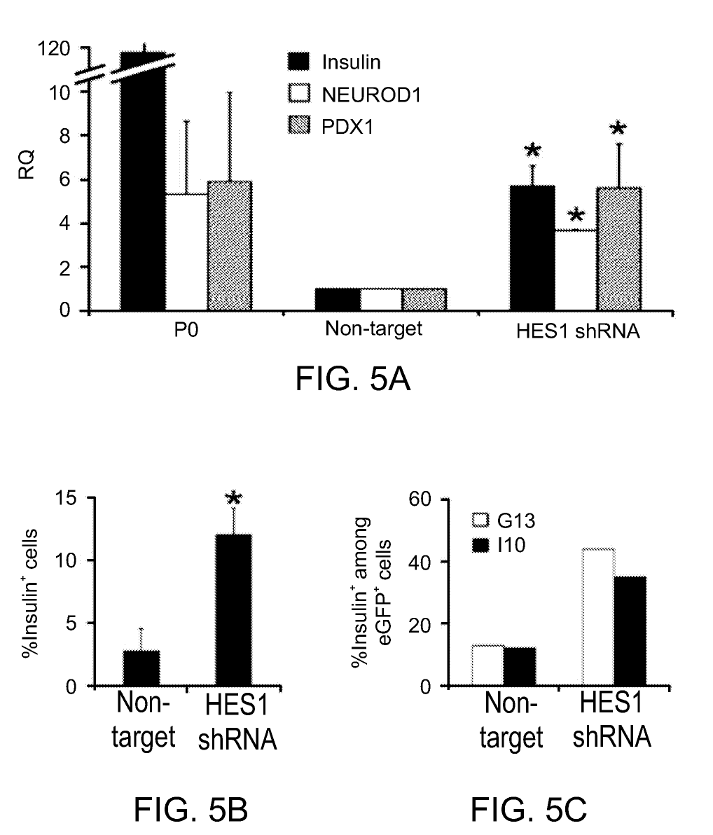

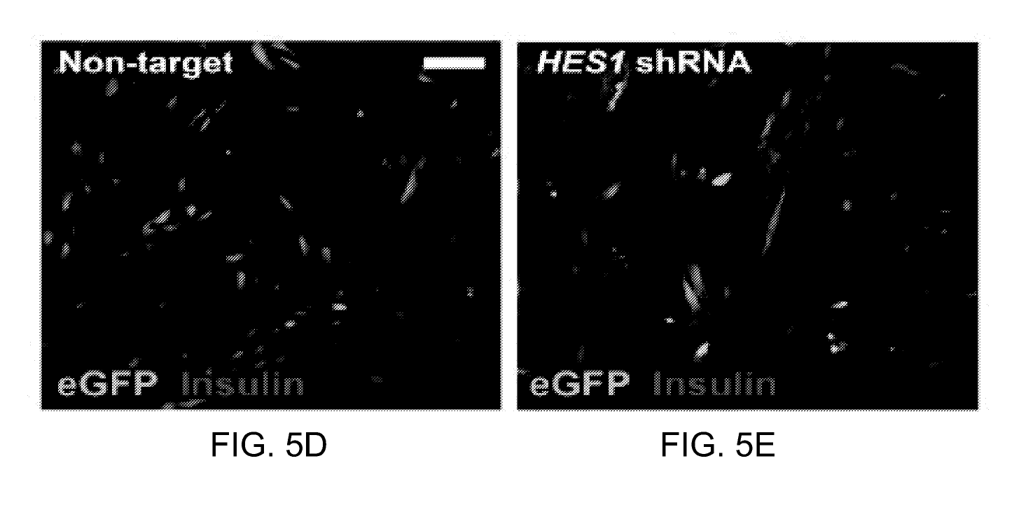

Inhibition of HES1 expression reduces beta-cell dedifferentiation: The lower HES1 levels in cells expressing HES1 shRNA resulted in a reduced rate of cell dedifferentiation, as manifested by higher levels of transcripts encoding differentiated beta-cell markers. Thus, levels of insulin transcripts were 5.7-fold higher (p<0.01), compared with cells infected with the control virus (FIG. 5A). Similarly, transcript levels for the beta-cell transcription factors PDX1 and NEUROD1 were 5.6-fold-(p<0.05) and 3.7-fold-(p<5.45×10⁻⁵) higher in cells expressing HES1 shRNA (FIG. 5A). The levels of PDX1 and NEUROD1 transcripts in cells expressing HES1 shRNA were comparable to those in primary islets. In contrast, the levels of insulin transcripts in cells expressing HES1 shRNA were still 20-fold lower, compared with those in primary islets. In agreement with the higher insulin mRNA levels, insulin immunostaining detected a 4-fold (p<0.016) higher number of insulin-positive cells in cultures expressing HES1 shRNA, compared with those treated with the control virus (FIGS. 5B-C). The fraction of insulin-positive cells among eGFP⁺ cells was also 3-fold higher in the presence of HES1 shRNA, indicating that fewer beta cells underwent dedifferentiation (FIG. 5B-E).

Discussion

These findings demonstrate that culture of dissociated adult human islet cells in serum-containing medium, which induces beta-cell dedifferentiation and replication, involves activation of elements of the NOTCH pathway. Transcript levels for NOTCH1-3 and HES1 were upregulated. In contrast, transcripts for NOTCH4, and the NOTCH ligands DELTA1, JAG1, and JAG2, were downregulated. These changes were initially observed in a mixed population of islet cells, which likely included contaminating duct and exocrine cells. Using virus-mediated cell-lineage tracing, the present inventors then determined that these changes occurred in beta cells. The upregulation of the NOTCH pathway correlated with cell dedifferentiation, as manifested by a dramatic decrease in insulin transcripts, and by cell entrance into the cell cycle, as manifested by downregulation of p57 transcripts and an increase in Ki67 staining. The findings at the RNA level were supported by immunostaining, which demonstrated a negative correlation between the presence of HES1 or NICD in the nucleus, and insulin expression, in eGFP⁺ cells, which marked their origin from beta cells. These in situ analyses also detected a positive correlation between p57 and insulin expression, confirming the view that beta-cell replication involves dedifferentiation.

The key role of HES1 in these events was revealed by inhibiting its upregulation with shRNA. In these cells, the decrease in p57 was prevented, and cell proliferation was greatly reduced. While cell dedifferentiation was not completely prevented, it was significantly inhibited, compared with cells in which HES1 upregulation was not repressed. This was manifested by higher levels of insulin transcripts and fraction of cells immunostaining for insulin, as well as transcripts encoding beta-cell transcription factors. These findings suggest that a partial cell dedifferentiation is independent of HES1 activity and cell replication, however induction of advanced dedifferentiation and cell replication requires HES1 upregulation. This interpretation is supported by the finding that the bulk of decrease in insulin mRNA occurs during the first week, thus preceding the peak in HES1 mRNA levels. It is therefore possible that the loss of most insulin is a precondition for beta-cell entrance into cell cycle in vitro.

Given the fact that upregulation of the NOTCH pathway in islet cell cultures followed cell dissociation into single cells, it is unlikely that it was triggered by a cell-associated ligand, as in the lateral inhibition model [Apelqvist A, et al., Nature 400:877-881, 1999]. Rather, it is possible that this pathway is activated in response to soluble serum components, as was demonstrated in a number of cultured cell types [Hirata et al., Science 298:840-843, 2002]. This possibility is supported by the present findings of decreased expression of NOTCH ligands in islet cell cultures concomitantly with HES1 upregulation. This is reminiscent of the low levels of NOTCH ligands in the embryonic pancreas cells expressing HES1, which are directed for further proliferation, rather than differentiation [Apelqvist A, et al., Nature 400:877-881, 1999].

Among the 4 members of the NOTCH family that were analyzed, NOTCH1, NOTCH2, and NOTCH3 transcripts were upregulated, while NOTCH4 transcripts were greatly downregulated. While expression of NOTCH1 and NOTCH2 was implicated in islet development, NOTCH3 and NOTCH4 expression was documented in mesenchymal and endothelial cells. Downregulation of NOTCH4 may reflect the elimination of a subpopulation in the original islet cell suspension, which does not attach well and is therefore not maintained in culture.

The wave of HES1 upregulation peaked within the first 2 weeks of culture and was downregulated thereafter. Nevertheless, the effects of HES1 were not reversed, as manifested by continuous replication of cells derived from dedifferentiated beta cells for up to 16 population doublings [Ouziel-Yahalom et al., Biochem Biophys Res Commun 341:291-298, 2006; Russ H A, et al (2008) Diabetes 57:1575 -1583]. The levels of p57 and insulin transcripts did not rebound thereafter, suggesting that their induction requires other signals, in addition to the decrease in the inhibitory effect of HES1. This finding suggests a transient role of HES1 upregulation that is limited to the initial adaptation of islet cells to culture, after which cell replication may continue in the presence of the low HES1 levels found in non-replicating cells.

### Example 2

### Effect of HES1 Inhibition on Redifferentiation of Dedifferentiated Human Islet Cells Expanded in Culture

Materials and Methods

Human islet cells were cultured for 4-5 weeks as described for Example 1. They were then infected with lentiviruses expressing HES1 (SEQ ID NO: 7) or nontarget shRNAs. Nine days following infection the cells were analyzed as described for Example 1. Primers used for RT-PCR analysis are described in Table 1, herein above,

Results

As seen in FIG. 6, HES1 shRNA caused a decrease in cellular HES1 protein levels and induced an increase in p57 levels. The increase in p57 was confirmed by

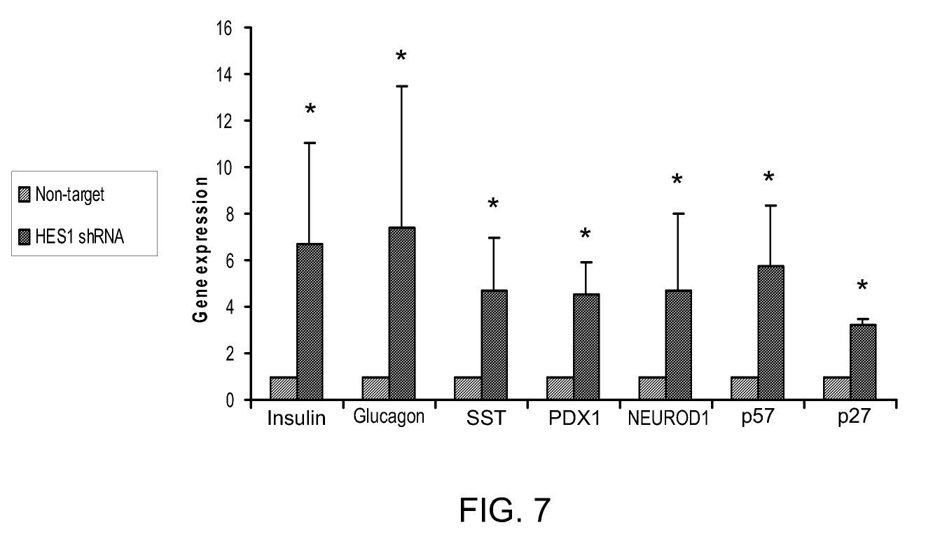

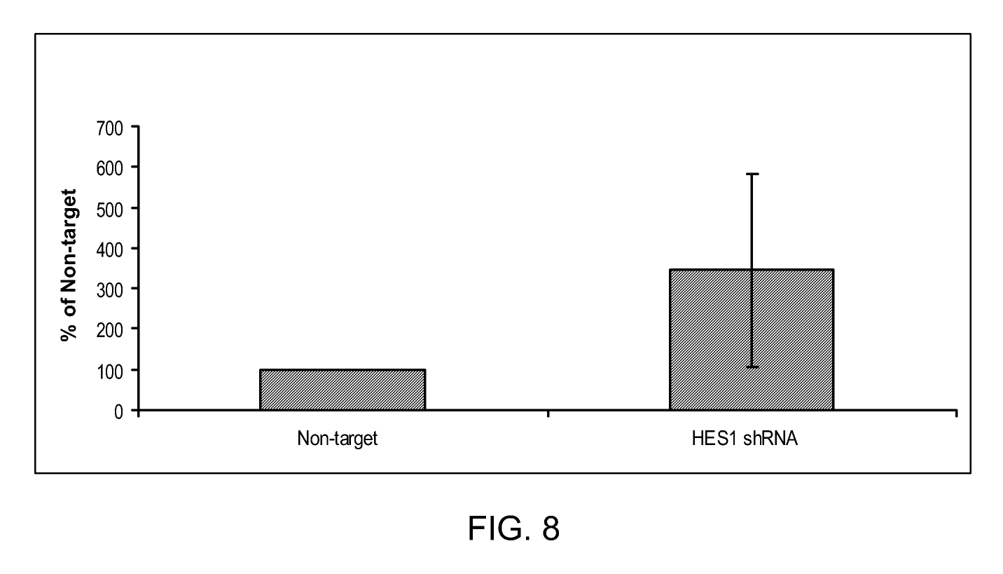

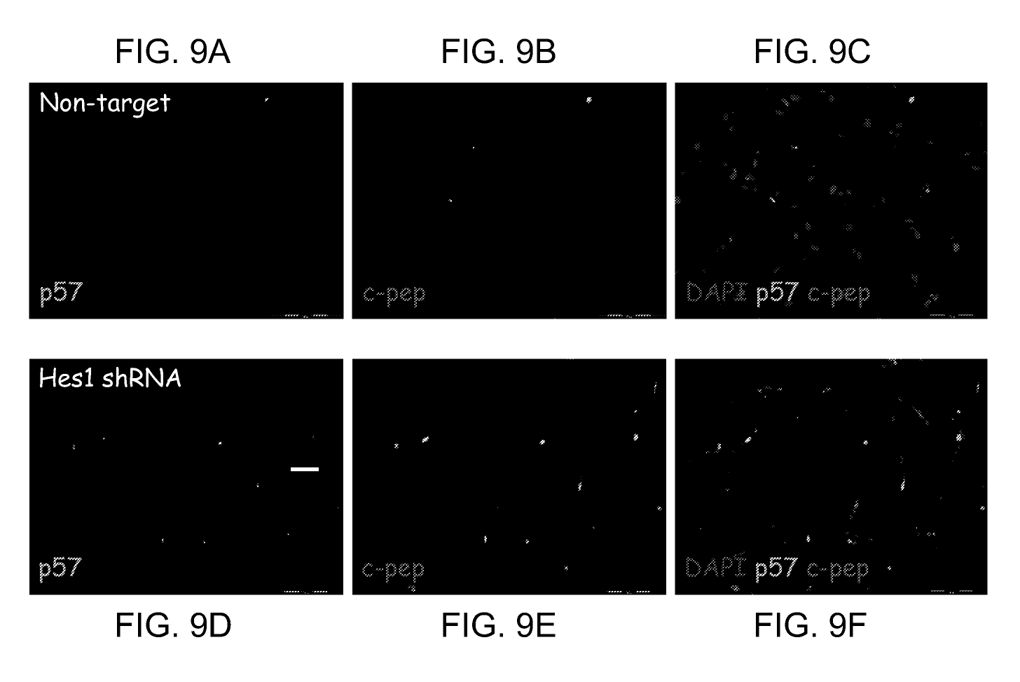

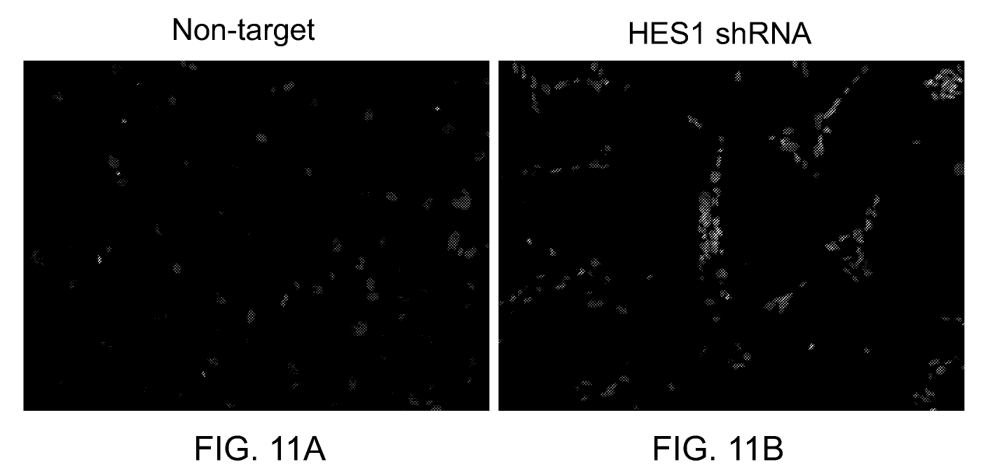

RNA analysis (FIG. 7) and immunostaining (FIGS. 9A-F). The RNA analysis also showed a significant increase in insulin transcripts, as well as transcripts encoding the beta-cell transcription factors PDX1 and NEUROD1. The increase in insulin expression was confirmed by insulin ELISA (FIG. 8) and immunostaining (FIGS. 9A-F). A noticeable morphological change occurred in cells expressing HES1 shRNA which tended to form clusters (FIGS. 11A-B).

These findings demonstrate a reproducible differentiating effect of HES1 shRNA in dedifferentiated human islet cells expanded in culture, manifested by an increase in expression of the cell cycle inhibitor p57 and markers of beta-cell differentiation, most notably insulin. Thus, it can be concluded that HES1 shRNA can be used for redifferentiation of dedifferentiated human islet cells following expansion in culture.

### Example 3

### In Vitro Proliferation of Cells Derived From Adult Human Beta Cells Revealed by Cell-Lineage Tracing

Materials and Methods

Lentivirus vector construction and virus production: The pTrip RIP405 nlsCRE DeltaU3 (RIP-Cre) vector was generated by removing with BamHI and XhoI the GFP coding region from the pTrip RIP405 eGFP DeltaU3 vector [Castaing M, et al., Diabetologia 48:709-719, 2005], which contains a fragment of the rat insulin II gene from −405 to +7 relative to the transcription start site. The resulting linearized plasmid was blunt-ended with DNA polymerase I Klenow fragment. The reading frame A Gateway cassette (Gateway Conversion Kit, Invitrogen) was next ligated to the blunt ended vector according to manufacturer instructions, generating a pTrip RIP405 rfa-Gateway DeltaU3 destination vector. The nlsCRE fragment was amplified by PCR from a plasmid [Thévenot E, et al., Mol Cell Neurosci 24:139-147, 2003] using the forward primer 5′CACCAGATCTATGCCCAAGAAGAAGAGG3′[SEQ ID NO: 1] and reverse primer 5′CTCGAGCTAATCGCCATCTTC3′ [SEQ ID NO: 2], and the resulting PCR product was cloned into the pENTR/D/TOPO plasmid (Invitrogen) to generate a nls-CRE entry plasmid (SEQ ID NO: 13). Both destination vector and entry clone were used for in vitro recombination using the LR clonase II system (Invitrogen) according to manufacturer instructions. The reporter vector was constructed by amplifying the loxP-DsRed2-loxP cassette by PCR from a plasmid using the forward primer 5′AATTCACTAGTGAACCTCTTC3′ [SEQ ID NO: 3] and the reverse primer 5′ GATCCGATCATATTCAATAA3′ [SEQ ID NO: 4]. The resulting PCR product was ligated into the blunt-ended BamHI site of the pTrip CMV eGFP DeltaU3 vector [Castaing M et al., Diabetologia 48:709-719, 2005], resulting in the pTrip CMV-loxP-DsRed2-loxP-eGFP DeltaU3 lentiviral vector (SEQ ID NO: 14). Virus particles were produced in 293T cells following vector cotransfection with the pCMVdR8.91 and pMD2.G plasmids. The culture medium was harvested 36-48 hours later. Islet cell culture and infection with viruses. Islet purity was determined by staining with dithizone. Islets were received 2-3 days following isolation. Islets from individual donors were dissociated into single cells and cultured in CMRL 1066 medium containing 5.6 mM glucose and supplemented with 10% FBS, 100 U/ml penicillin, 100 μg/ml streptomycin, 100 μg/ml gentamycin, and 5 μg/ml amphotericine B as described [Ouziel-Yahalom et al., Biochem Biophys Res Commun 341:291-298, 2006]. Following 1-2 days in culture cells were washed with PBS and infected with a 1:1 mixture of the 2 viruses at MOI 3:1 in CMRL containing 8 pg/ml polybrene overnight. βTC-tet cells were infected at MOI 1.5:1. The medium was then replaced with regular culture medium. Cells were refed twice a week and split 1:2 once a week. Conditioned medium was obtained 2-3 days following the last change of medium, centrifuged at 1000 rpm for 4 minutes, filtered with a 0.22-Pm filter, and stored at −20° C. Mouse islets were isolated from 5-month-old BALB/c mice by collagenase infusion through the bile duct and treated similarly to the human islets.

Cell sorting: Labeled cells were sorted using a FACS Aria cell sorter (Becton Dickinson, San Jose, Calif.) with a fluorescein isothiocyanate (FITC) filter (530/30 nm) for eGFP, and a Pe-Texas Red filter (610/20 nm) for DsRed2. Dead cells stained with 7-amino actinomycin D (7-AAD, Invitrogen) were excluded using a PerCP-Cy5.5 filter (695/40 nm). Islet cells infected with the reporter virus alone or with the Turbo GFP virus (Sigma) were used for setting the gating for DsRed2 and eGFP, respectively.

Immunofluorescence: Cells seeded on sterilized coverslips were washed with PBS and fixed with 4% paraformaldehyde for 10 minutes at room temperature. For nuclear antigens, slides were permeabilized for 10 minutes with 0.25% NP40. Cells were blocked for 20 minutes with 5% fetal goat serum, 1% bovine serum albumin and 0.2% saponin and incubated for 1 hour with the primary antibodies, diluted in blocking solution. Cells were then washed and incubated for 40 minutes with the secondary antibodies. Images were taken using a Zeiss LTM 200 Apotome. Images of fluorescent living cells were taken with a long-distance objective on a Zeiss LTM 200 microscope. Expression of eGFP was detected using mouse anti-GFP (Chemicon, 1:500) or rabbit anti-GFP (Invitrogen, 1:1000). DsRed2 was visualized by endogenous fluorescence. Other primary antibodies used: mouse anti-insulin (Sigma, 1:1000), guinea pig anti-insulin (Dako, 1:1000), mouse anti-human C-peptide (Biodesign, 1:200), mouse anti-glucagon (Sigma, 1:2000), rabbit anti-somatostatin (Dako, 1:200), rabbit anti-pancreatic polypeptide (Dako, 1:200), rabbit anti-amylase (Sigma, 1:200), mouse anti-cytokeratin 19 (Sigma, 1:50), mouse anti-Cre (Chemicon, 1:2000), rabbit antihuman Ki67 (Zymed, 1:50), and rabbit anti-mouse Ki67 (Neo markers, 1:100). Secondary antibodies used: anti-mouse, anti-rabbit-, or anti guinea pig-AMCA (Jackson, 1:200), -Cy2 (Biomed, 1:200), -Cy3 (Biomed, 1:300), and -Alexa 700 (Invitrogen, 1:100). DNA was stained with DAPI (Sigma).

DNA analysis: Cell genomic DNA was isolated using the High Pure PCR template preparation kit (Roche Molecular Biochemicals, Mannheim, Germany). PCR analysis of the integrated reporter vector was performed with the forward primer 5′AACAACTCCGCCCCATTG3′ (SEQ ID NO: 5) and the reverse primer 5′CTCCTCGCCCTTGCTCAC3′ (SEQ ID NO: 6). This primer pair amplifies an 1129-bp fragment from the original sequence and a 338-bp fragment from the recombined sequence. PCR products were resolved on a 1.5%-agarose gel containing ethidium bromide.

RNA analysis: Total RNA was extracted using High Pure RNA isolation kit (Roche). Total RNA was amplified using Ovation™ Aminoallyl RNA Amplification and Labeling System (Nugen). cDNA quantitation was performed using the following Assay-on-Demand kits (Applied Biosystems): insulin, Hs_{—}00355773_m1; PDX1, Hs_{—}00426216_m1; NKX2.2, Hs_{—}00159616_m1; glucagon, Hs_{—}00174967_m1; NEUROD1, Hs_{—}00159598_m1; NKX6.1, Hs_{—}00232355_m1; glucokinase, Hs_{—}00175951_m1; PC1/3, Hs00175619_m1; PC2, Hs_{—}00159922_m1; GLUT2, Hs_{—}01096908_m1; PTF1a, Hs_{—}00603586_g1; HNF4a, Hs_{—}00230853_m1; PAX4, Hs_{—}00173014_m1; PAX6, Hs_{—}00240871_m1; HNF6, Hs00413554_m1; NGN3, Hs00360700_g1; RPLP0, Hs_{—}99999902_m1. All reactions were done in triplicates. The results were normalized to human large ribosomal protein P0 cDNA (RPLP0).

Immunoblotting: Total protein was extracted by incubating cells for 10 minutes in 1% NP40 containing a protease inhibitor cocktail. Protein concentration was determined using the BCA Protein Assay Kit (Pierce). 40 pg protein were resolved on a SDS-PAGE gel. The gel was electroblotted onto Immobilon-P Transfer Membrane (Milipore), followed by incubation with rabbit anti-cleaved poly(ADP-ribose) polymerase (PARP) (Cell Signaling, 1:1000) or rabbit anti-p21 (Santa Cruz, 1:200). Goat anti-actin (Santa Cruz, 1:1000) was used to monitor gel loading. The bound antibody was visualized with the appropriate horseradish peroxidase-conjugated anti-IgG and SuperSignal West Pico Chemiluminescent Substrate (Pierce). Cells treated with 1 pM staurosporine for 6 hours were used as positive control.

Results

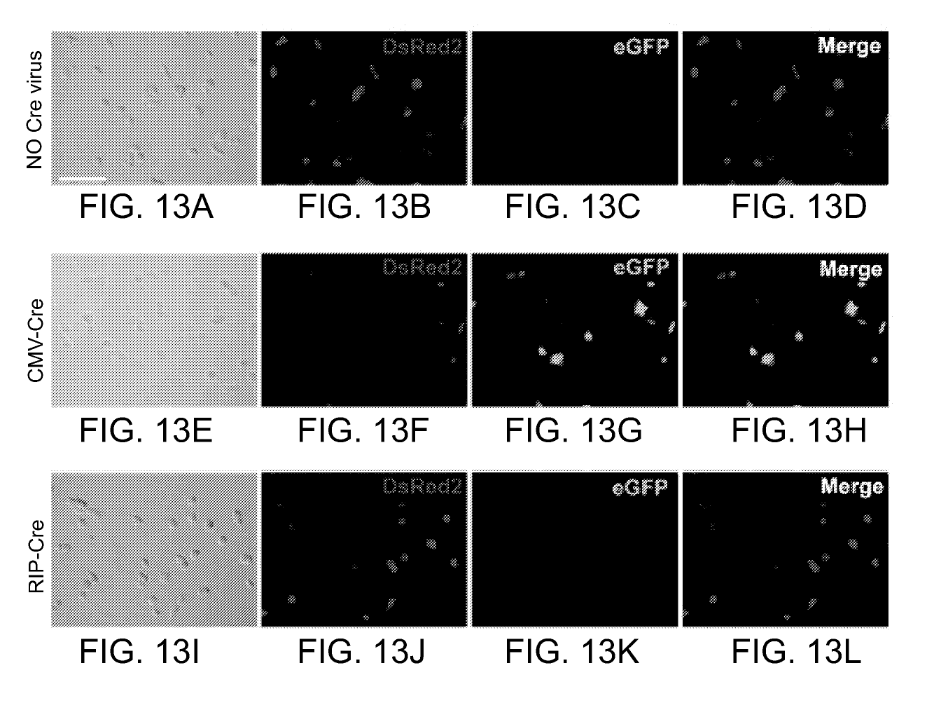

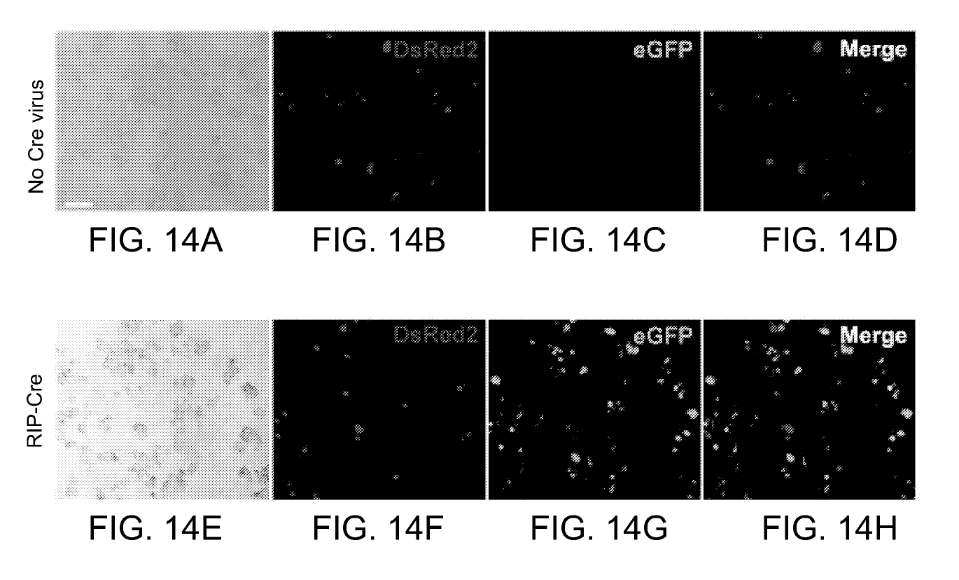

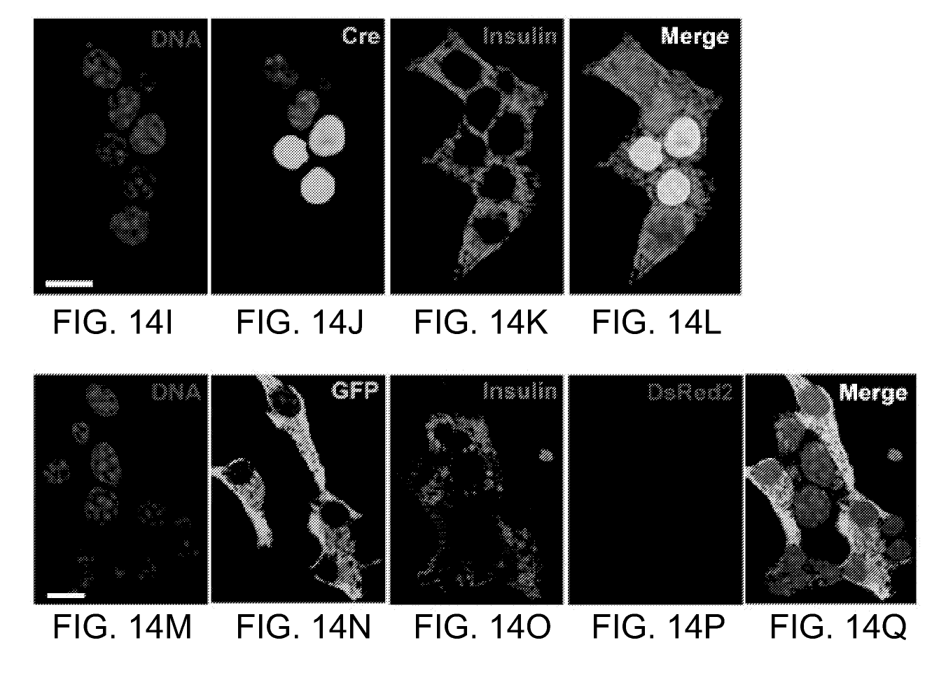

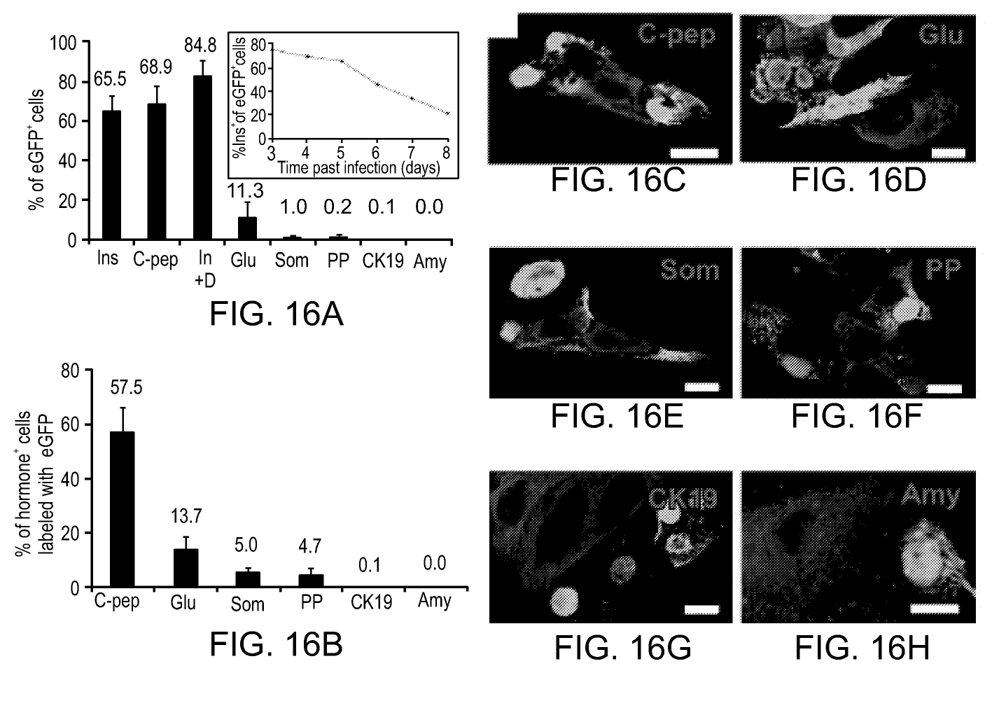

The labeling approach is based on cell infection with a mixture of 2 lentivirus vectors, one expressing Cre recombinase under control of the insulin promoter (RIP-Cre; FIG. 12A), and the other a reporter cassette with the structure CMV promoter-loxP-DsRed2-loxP-eGFP (FIG. 12B). The latter virus expresses the fluorescent marker DsRed2 in all cells infected by it, while expression of enhanced green fluorescent protein (eGFP) is blocked. Removal of the DsRed2 coding sequence between the 2 loxP sites in beta cells infected by both viruses is expected to eliminate DsRed2 expression specifically in these cells and activate instead GFP expression, which should allow continuous tracking of beta-cell fate after insulin expression is lost. Human beta cells were shown to maintain insulin expression during the initial days in culture [Ouziel-Yahalom et al., Biochem Biophys Res Commun 341:291-298, 2006], thereby providing a window of time for RIP-Cre expression. Non-beta cells infected with both viruses are expected to express only DsRed2. The specificity of the labeling system was evaluated using the mouse beta-cell line βTC-tet, and 293T cells as a negative control. No eGFP+ cells were detected in 293T cells infected with the reporter virus alone or with a mixture of the 2 viruses (FIGS. 13A-L), demonstrating a tight inhibition of eGFP expression in the absence of RIP-Cre expression. This finding demonstrates the lack of non-specific RIP-Cre expression, such as could potentially be caused by virus integration next to a strong promoter, as well as a lack of GFP expression in the presence of the DsRed2 region. In contrast, Cre expression under CMV promoter resulted in efficient activation of eGFP expression. Presence of both eGFP and DsRed2 proteins in the same cells, manifested by yellow color, likely reflects the relatively long half-life of the DsRed2 protein (4.5 days), which may still be detected 1-2 weeks following loss of the DsRed2 gene and activation of eGFP expression. In βTC-tet cells infected with the reporter virus alone 61.2% of the cells became DsRed2+, indicating a high efficiency of cell infection with this vector (based on >1000 cells counted in micrograph images; FIGS. 14A-H). In cells infected with the RIP-Cre virus alone, 100% of Cre+ cells were also insulin positive (FIGS. 14I-L). Given the infection efficiency with a single virus, in cells infected with both viruses the incidence of eGFP+ and DsRed2+cells is expected to be 37.4% (0.612×0.612) and 23.8% (0.612-0.374), respectively. The observed incidence was 32.3% and 30.6% for eGFP+ and DsRed2+, respectively (based on >1000 cells counted in micrograph images; FIGS. 14A-H). All eGFP+ cells (100%) stained for insulin (FIGS. 14M-Q). Islets were isolated from 15 human donors, 10 males and 5 females, aged 17-60 (mean age 46±12), with a purity ranging between 70-90% (mean 78±6%), as determined by staining with dithizone. Islets from each donor were dissociated into single cells and cultured as described [Ouziel-Yahalom et al., Biochem Biophys Res Commun 341:291-298, 2006]. Since human islets were reported to contain on average 55% insulin-positive cells, these preparations were expected to contain <43±3% insulin-positive cells at the time of isolation (0.55×0.78). After 1-2 days in culture the cells were infected with the reporter virus alone, or with a mixture of the 2 viruses. At the time of infection the number of insulin-expressing cells was expected to be lower, compared with their number immediately following isolation, due to rapid dedifferentiation during the 2-3 days of shipment and 1-2 days of initial culture [Ouziel-Yahalom et al., Biochem Biophys Res Commun 341:291-298, 2006]. Cells infected with the reporter virus alone showed DsRed2 expression in 68.2±11.0% of the cells (based on flow cytometry analysis of cells at passages (P) 2-6, derived from 5 donors, 2-10×10³ cells per sample; FIGS. 15A-H), demonstrating a high efficiency of cell infection with this vector. <0.08% of the cells showed an eGFP signal, indicating a low leakiness of eGFP expression in these cells in the absence of Cre expression. In islet cells infected with both viruses, 17.9±6.8% of the cells were labeled with eGFP, while 41.5±7.4% of the cells were DsRed2-positive (based on flow cytometry analysis of cells at P2-5, derived from 5 donors, 2-10×103 cells per sample, ranging between 8.7-31.5% eGFP+ cells; FIGS. 15A-H). The calculated labeling efficiency, based on the efficiency of infection with a single virus (68.2%) and the expected beta-cell content (<43±3%) given the islet purity, was 20% (0.682×0.682×0.43). The observed incidence of eGFP+ cells, 17.9±6.8%, is not far from this value. Similarly, the calculated labeling efficiency for DsRed2+ cells was 48.2% (68.2−20%), while the observed value was 41.5±7.4%. The ˜40% unlabeled cells likely include uninfected cells, as well as cells infected by the RIP-Cre virus alone, while the DsRed2+cells represent non-beta cells infected by the reporter virus alone or both viruses, and beta cells infected by the reporter virus alone. Among eGFP+ cells, 65.5%±7.1% were insulin-positive, and 68.9±8.9% were human C peptide-positive, as judged by immunostaining following 5-6 days in culture (FIG. 16A). This was the earliest time point (4-5 days following infection) at which eGFP could be clearly detected in live cells. A weak eGFP signal was detected earlier than 4-5 days post infection. However, it took a longer time for a strong signal to appear, probably due to accumulation of higher levels of eGFP in the cells. Scoring of cells was only performed after a strong, unequivocal, signal appeared. The insulin-negative eGFP+ cells likely reflect a rapid loss of insulin content between the time of gene recombination and immunostaining (FIG. 16A, inset). To further verify this possibility, cells were incubated following viral infection with 10 pM diazoxide, an inhibitor of insulin release. This treatment resulted in an increase in the fraction of eGFP+ cells that were insulin-positive to 84.8±5.8% (FIGS. 16A-B). It is possible that a higher concordance between insulin and eGFP could have been achieved by performing the labeling in the presence of lower glucose concentrations. However, the present inventors aimed at performing the labeling at 5.6 mM glucose, a concentration that was optimized for cell proliferation. The rate of insulin content loss at this concentration was expected to be moderate. Since Cre expression could be detected in the cells at an earlier time point following viral infection, compared with eGFP, the concordance between Cre and insulin expression was analyzed 36-hours post-infection. The analysis revealed that 96.2±0.6% of the Cre+ cells were insulin-positive (based on >1,000 cells counted from each of 3 donors) (FIGS. 15I-L). The efficiency of beta-cell labeling, as judged by the percent of C peptide-positive cells labeled with eGFP 4-5 days post-infection, was 57.5±8.9% (FIG. 16B).

To evaluate the incidence of non-specific labeling of other pancreatic cells, the infected cells were stained with antibodies for 3 other islet hormones, as well as for amylase, a marker of pancreatic exocrine cells, and CK19, a marker of pancreatic duct cells. 11.3±7.6% of the eGFP+ cells were stained for glucagon, accounting for 13.7±4.7% of glucagon-positive cells (FIGS. 16A-B). 1.0±0.6% of the eGFP+ cells were stained for somatostatin, while 1.2±0.3% were pancreatic polypeptide (PP)-positive, accounting for 5.0±1.8% and 4.7±2.1% of the cells staining for each hormone, respectively. Human islets were reported to contain on average 38% glucagon-positive and 10% somatostatin-positive cells, respectively (Cabrera O et al., Proc Natl Acad Sci USA 103:2334-2339, 2006). Co-staining with insulin showed that a large part of eGFP+ cells expressing other islet hormones co-expressed insulin, indicating that their labeling by eGFP was specific, while the remainder may have expressed insulin at the time of viral infection, but had lost its expression during the time between infection and staining. Co-expression of islet hormones has been documented in human fetal islets, but not in adult islets. As shown in FIGS. 16A-B, <0.1% of amylase- or CK19-positive cells were stained with eGFP. Thus, the bulk of insulin-negative eGFP+ cells did not stain for any of the other markers analyzed. All cells stained for the mesenchymal marker vimentin (data not shown), which was shown to be activated in all cultured islet cells (Ouziel-Yahalom et al., Biochem Biophys Res Commun 341:291-298, 2006). Infection of cells at P7-8, at which no insulin-positive cells could be detected, with both viruses did not result in cell labeling with eGFP, providing further evidence for the dependence of transgene recombination on insulin expression (data not shown).

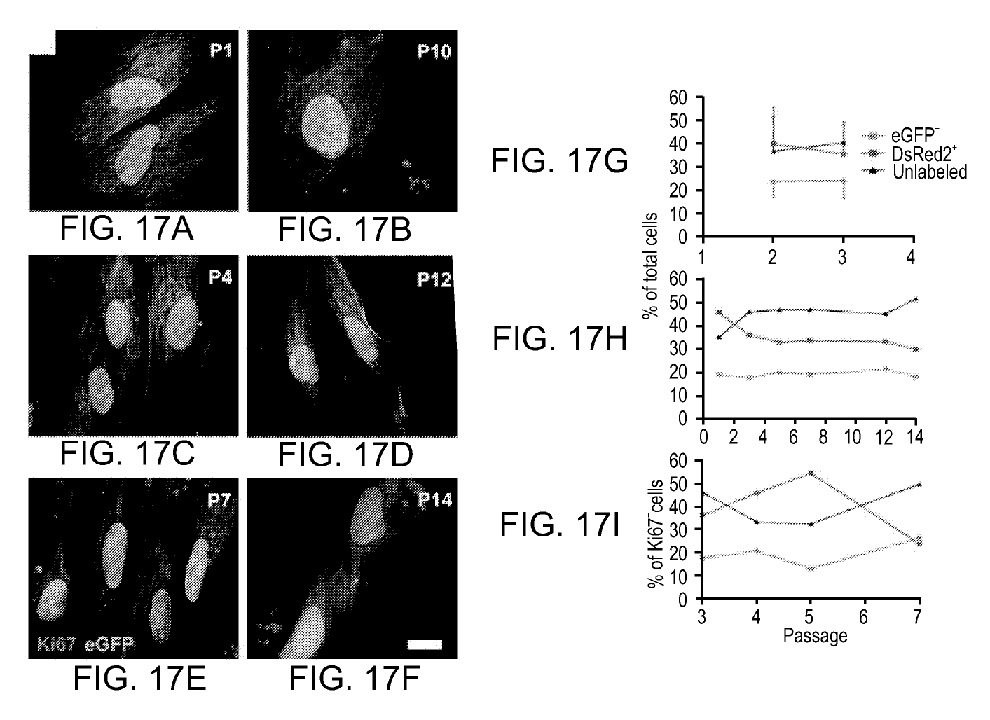

Taken together, these findings demonstrate that the labeling of beta cells with eGFP in this system was efficient and specific. The eGFP+ cells were followed during continuous culture. As previously reported (Ouziel-Yahalom et al., Biochem Biophys Res Commun 341:291-298, 2006), the cultured islet cells replicated with an average doubling time of 7 days, as judged by cell counting, for up to 16 population doublings, before undergoing senescence. Plating efficiency was high at all passages, and no significant cell mortality was detected. As seen in FIGS. 17A-F, replicating eGFP+ cells could be detected during the entire expansion period, as manifested by staining for Ki67 at P2, P4, P6, P12, and P14. At P2, 31.1±5.0% of eGFP+ cells were stained for Ki67. The proportion of eGFP+ cells among cultured cells remained stable during this expansion period (FIGS. 17G-I), demonstrating that the doubling rate of the eGFP+ cells was similar to that of eGFP-negative cells. Overall, replicating eGFP+ cells were detected in multiple passages of islet cell cultures from 15/15 adult donors studied. The data was highly reproducible among all donors studied, and no age- or gender-related differences were noted.

Taken together, these findings demonstrate that dedifferentiated beta cells survive and replicate for a considerable number of population doublings, and can be traced during this period by following the eGFP label. These findings are in contrast to the inability to demonstrate in-vitro proliferation of mouse beta cells labeled by transgenic approaches. The present inventors therefore utilized the viral labeling strategy to evaluate mouse beta-cell proliferation under conditions similar to those used for human cells.

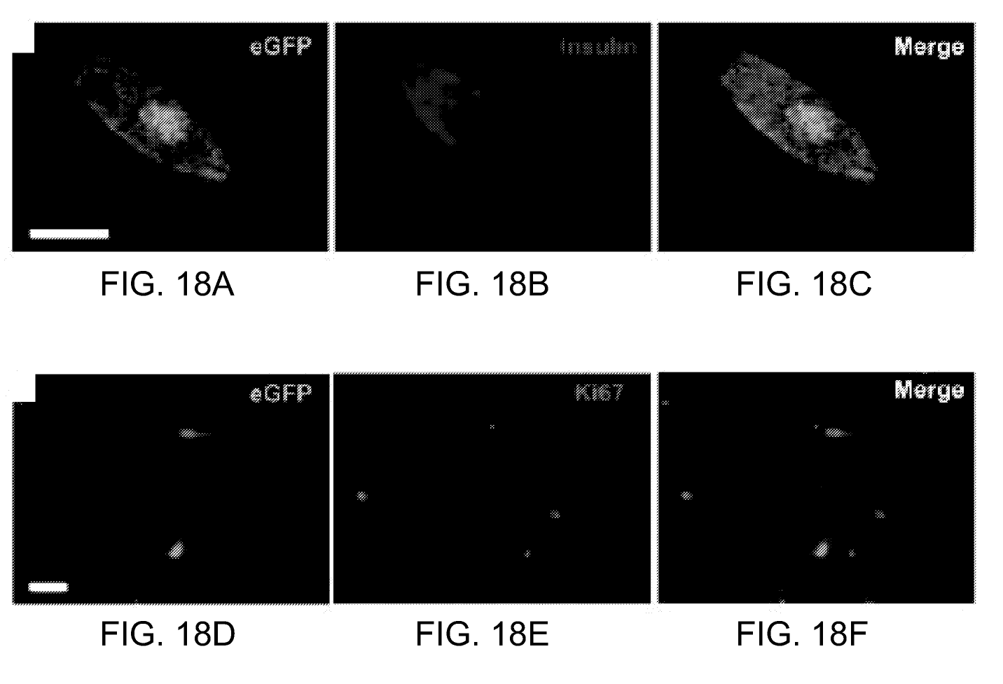

Isolated mouse islets were trypsinized and infected with the 2 lentiviruses. The labeling efficiency of the mouse cells was in the range of that of the human cells: 22.6% of insulin-positive, and 7.4% of all cells, as quantitated 5 and 10 days following viral infection, respectively (FIGS. 18A-F). When these cultures were analyzed 11-14 days post infection, 0.5% of eGFP+ cells were Ki67+, compared with 31% of human eGFP+ cells at P2 (FIGS. 18A-F). By day 20 post-infection, the incidence of eGFP+ cells in the population decreased to 1.37%, compared with stability around 20% in the human cell culture, indicating that the culture was increasingly dominated by proliferating cells from a non-beta-cell origin, in accordance with previous findings in transgenic mouse islet cell cultures (Weinberg et al., Diabetes 56:1299-1304, 2007). These findings confirm the species difference between mouse and human beta cell proliferation under the present culture conditions.

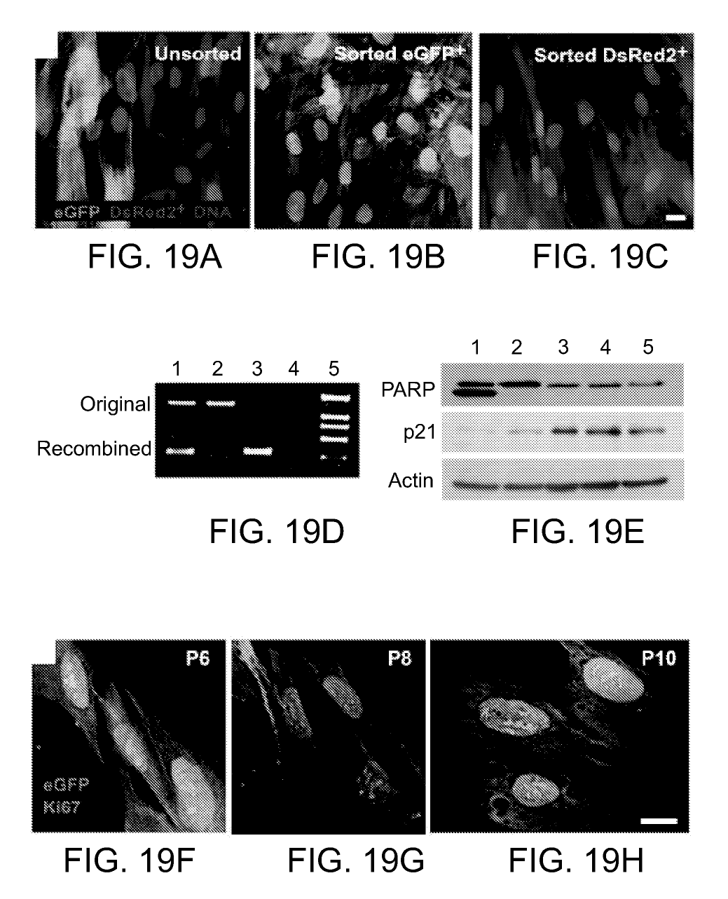

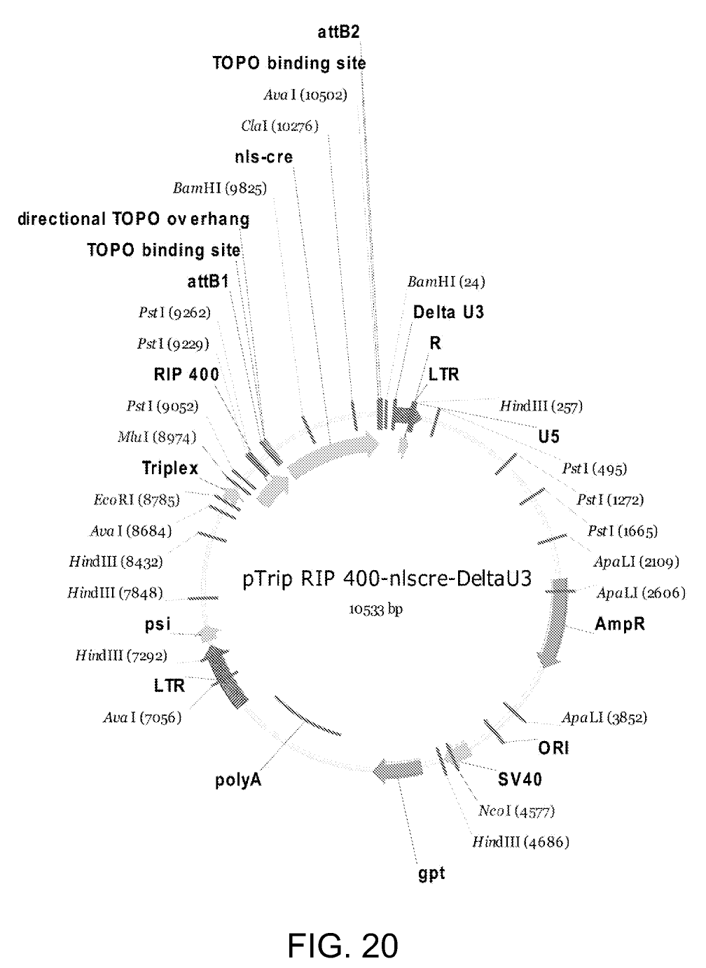

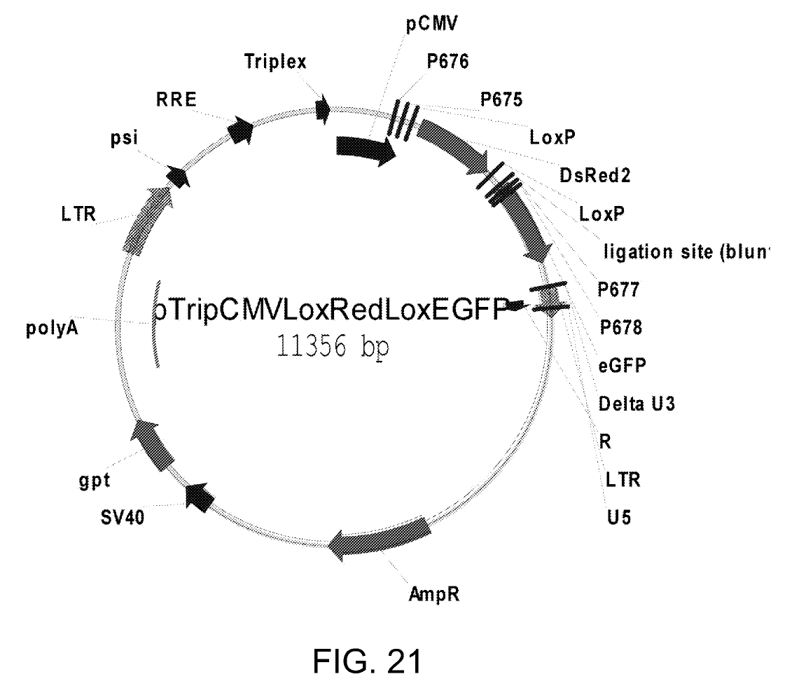

eGFP+ and DsRed2+ cells from the human islet cultures were sorted by FACS and analyzed for transgene recombination and gene expression (FIGS. 19A-C). DNA analysis of the sorted cells detected in eGFP+ cells only the recombined gene, while DsRed2+ cells contained only the original gene (FIG. 19D). Quantitative RT-PCR analysis of RNA extracted from eGFP+ and DsRed2+ cells at P2 and P12 documented the enrichment of beta-cell markers in eGFP+, compared with DsRed2+ cells, at P2, and the dedifferentiation of eGFP+ cells by P12 (Table 2, herein below). However, transcript levels in eGFP+ cells at P2 were 5-37-fold lower, compared with the unsorted islet cell population at P0. Glucagon transcripts were detectable in eGFP+ cells, confirming the immunofluorescence results (FIGS. 16A-H), however they were enriched in DsRed2+ cells. Low levels of insulin, PC2, glucagon, and PAX6 transcripts were still detectable in eGFP+ cells at P12, however all other beta-cell transcripts were not detected at this stage. The fact that not all beta-cell transcripts were enriched to the same extent by cell sorting, when comparing eGFP+ and DsRed2+ cells at P2, could result from different abundance of transcripts of different genes, which may lead to cDNA amplification bias. Transcripts for PTF1a, HNF6, and NGN3, which are expressed during pancreas development, were not detected in any of the samples.

TABLE 2
Quantitative RT-PCR analysis of RNA from eGFP⁺ and DsRed2⁺ cells sorted
at the indicated passages.

| | | | | | eGFP⁺/ |
|---|---|---|---|---|---|
| | eGFP⁺ | | DsRed2⁺ | | DsRed2⁺ at |
| Gene | P2 | P12 | P2 | P12 | P2 |
| Insulin | 2.6688 ± 0.2894 | 0.0003 ± | 0.0634 ± | ND | 42.13 |
| | | 8.33E−05 | 3.5E−03 | | |
| GK | 3.9464 ± 1.5168 | ND | 0.0865 ± | ND | 46.29 |
| | | | 0.0234 | | |
| PDX1 | 5.3896 ± 4.1185 | ND | ND | ND | — |
| NEUROD1 | 10.2569 ± 0.6220 | ND | 2.2745 ± | ND | 4.52 |
| | | | 0.1872 | | |
| NKX2.2 | 8.2412 ± 1.6133 | ND | 2.9353 ± | ND | 2.87 |
| | | | 0.8095 | | |
| NKX6.1 | 6.9992 ± 0.6214 | ND | 0.8264 ± | ND | 8.56 |
| | | | 0.1109 | | |
| HNF4α | 4.8116 ± 0.5688 | ND | 0.6863 ± | ND | 7.29 |
| | | | 0.2045 | | |
| PAX4 | 4.2179 ± 1.8572 | ND | ND | ND | — |
| PAX6 | 20.1707 ± 2.5206 | 0.1231 ± | 4.0884 ± | 1.74E−03 ± 1.11E−03 | 4.94 |
| | | 0.0126 | 0.3867 | | |
| PC1/3 | 7.4222 ± 0.3399 | ND | 0.0668 ± | ND | 112.20 |
| | | | 0.0135 | | |
| PC2 | 5.3809 ± 0.4646 | 2.59E−04 ± | 2.1508 ± | ND | 2.50 |
| | | 7.84E−05 | 0.1425 | | |
| GLUT2 | 13.7706 ± 2.8759 | ND | ND | ND | — |
| Glucagon | 5.6185 ± 2.2397 | 1.1E−03 ± | 9.1887 ± | 2.47E−03 ± 7.54E−04 | 0.61 |
| | | 5.29E−04 | 3.7820 | | |
| Vimentin | 154 ± 27 | 465 ± 193 | 181 ± 24 | 551 ± 111 | 0.84 |

The data are mean ± SD (n = 3) relative quantification (RQ) normalized to human RPLP0 and expressed as % of the levels in unsorted islet cells at P0.
ND, not detectable.

The replication capacity of sorted eGFP+ cells were then analyzed. Compared with a doubling time of 7 days in the mixed population, eGFP+ cells sorted at P8 with a purity >90% grew very slowly and doubled approximately once in 4 weeks. Supplementing the culture medium with 50% medium conditioned for 2 days by the mixed islet cell population at P0, or for 3 days by P10 DsRed2+ cells sorted at P8, resulted in a decrease in the doubling time to 9 and 10 days, respectively. The doubling time of FACS-sorted DsRed2+ cells remained 7 days. eGFP+ cells sorted at P8 could be propagated for 8 population doublings in the presence of conditioned medium before ceasing to replicate, representing a 256-fold expansion. Growth arrest was not associated with detectable apoptosis, as judged by immunoblotting analysis for cleaved poly(ADP-ribose) polymerase (PARP) (FIG. 19E). In contrast, p21, a protein involved in replicative senescence induced by telomere shortening, was upregulated in cells in the terminal passages (FIG. 19E). In contrast to high-purity sorted eGFP+ cells, sorted eGFP+ cells with a purity <80% grew equally well in the presence or absence of conditioned medium, indicating that the residual non-beta cells remaining in those preparations were sufficient for conditioning the medium. eGFP+ cells sorted at P5-8 could be stained for Ki67 at multiple subsequent passages (FIG. 19F-H). Taken together these results suggest that the eGFP-negative cells secrete growth factor(s) needed for proliferation of eGFP+ cells, and that the sorted eGFP+ cells become senescent around P16. This number of passages represents a theoretical overall expansion of 65,536-fold.

Discussion

The findings of this example provide for the first time direct evidence for survival and dedifferentiation of cultured adult human beta cells. In addition, they demonstrate that the dedifferentiated cells can significantly proliferate in vitro. Dedifferentiation may be a precondition for beta-cell proliferation in vitro, as evidenced by the scarcity of insulin+/Ki67+ cells in early passages of human islet cell cultures. However, dedifferentiation may not be sufficient for inducing beta-cell proliferation, as evidenced by the lack of replication of dedifferentiated mouse beta cells. The ability to purify human beta cells following genetic labeling in vitro will allow detailed studies of the molecular mechanisms involved in these two processes. In addition to replicating cells derived from beta cells, the islet cell cultures contain replicating cells which are not labeled with eGFP. Some of these cells may also be derived from beta cells, which were infected with only one or none of the 2 viruses. However, the majority of these cells are likely to be derived from other cellular origins, such as connective tissue in the islets or contaminating ductal tissue in the islet preparation. Nevertheless, the present findings show that cells derived from beta cells can be isolated and expanded in the absence of other cell types present in the islet cell culture, provided that their culture medium is supplemented with medium conditioned by non-beta cells. The factors released by these cells, which affect beta-cell growth, are of great interest, and the labeling system provides a convenient assay for their characterization. This system is also suitable for high-throughput screening of compound libraries for identification of agents which may further stimulate replication of the dedifferentiated beta cells in culture, as well as induce redifferentiation of the expanded cells.

The present work demonstrates the feasibility of cell-specific labeling of cultured primary human cells, using a genetic recombination approach that was previously restricted to transgenic animals. Despite the rapid dedifferentiation of beta cells in culture, virus integration into the genome and expression of the Cre recombinase under a beta-cell-specific promoter are apparently fast enough to allow efficient DNA recombination before dedifferentiation occurs, resulting in a remarkable efficiency of beta-cell labeling with this system (57.5±8.9% of the C-peptide-positive cells following 5-6 days in culture). It should be noted that the cell-specificity of this system relies on the use of a fragment of the regulatory region of the rat insulin II gene (−405 to +7 relative to the transcription start site), which is expected to allow Cre expression only in beta cells. However, while this region was shown in numerous studies to contain the major regulatory elements required for beta-cell-specific expression, it is possible that additional elements in the intact insulin gene locus are involved in determining tight cell-specificity. Thus, it can not be excluded that the absence of such elements in the RIP-Cre construct may result in its expression in a broader range of cells than bona fide mature beta cells.

Previous work has shown that proliferating cells expanded from cultured adult human islets contain cells expressing mesenchymal markers. Initially it was suggested that these cells originated from beta cells through epithelial-to-mesenchymal transition (EMT). Recent work has documented the expression of mesenchymal stem cell (MSC) markers on these cells, and their ability to differentiate into osteocytes and adipocytes, however their cellular origin has not been rigorously established. Using the labeling system described here it should be possible to determine if these cells are generated from beta cells by EMT or represent MSCs originally present in the islets.

The proliferation of dedifferentiated human beta cells in culture is in contrast with the inability to demonstrate such capacity in mouse beta cells cultured under the same conditions. It is possible that the culture media employed in this and previous studies with mouse islet cell cultures lack components needed for a significant expansion of mouse beta cells, while they are supportive for human beta-cell proliferation. Further investigation of this difference between mouse and human beta cells may provide new insights into the mechanisms that regulate beta-cell replication.

### Example 4

### Effect of HES Down-Regulation on B Cell Sorted Cells

Materials and Methods

Islet cell culture: Islets were received 2-3 days following isolation. Islets from individual donors were dissociated into single cells and cultured in CMRL 1066 medium containing 5.6 mM glucose and supplemented with 10% fetal bovine serum (FBS), 100 U/ml penicillin, 100 μg/ml streptomycin, 100 μg/ml gentamycin, and 5 μg/ml amphotericine B as described [Ouziel Yahalom et al., Biochem Biophys Res Commun 341:291-298, 2006]. The cultures were fed twice a week and split 1:2 once a week.

HES1 inhibition and lineage tracing: HES1 shRNA (TGGCCAGTTTGCTTTCCTCAT)—SEQ ID NO: 7 and a non-target shRNA, cloned in pLKO.1 lentiviral vector, were obtained from the RNAi Consortium (Sigma-Aldrich). Virus was produced in 293T cells following cotransfection with the pCMVdR8.91 and pMD2.G packaging plasmids. The culture medium was harvested 48 hours later. Adult human islet cells were labeled with eGFP (as described for Examples 1-3), sorted at passage 2, and expanded for a total of 7 population doublings. They were then infected with HES1 or nontarget shRNA viruses, and 4 days later trypsinized and shifted to serum free medium (SFM). RNA was extracted following 4 more days and analyzed by qRT-PCR.

RNA analyses: As described for Example 1.

Immunofluorescence: As described for Example 1.

Statistical Analysis: Significance was determined using Student's t-test.

Results

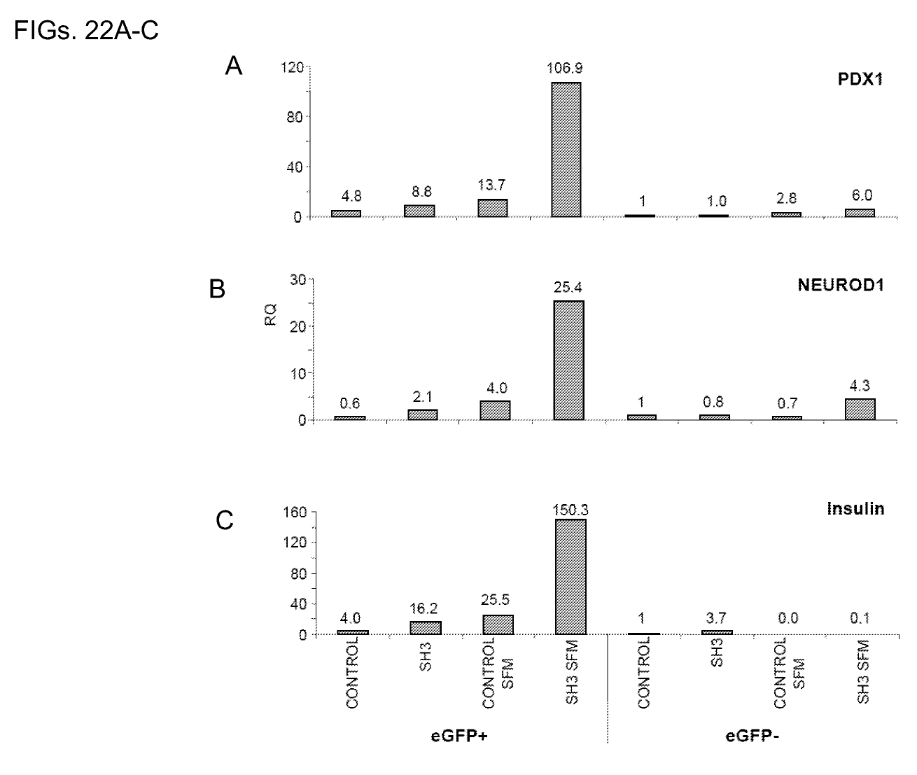

Beta-cell labeling with eGFP during the first week of culture, followed by sorting of eGFP⁺ cells, demonstrated that the morphological changes in response to HES1 shRNA were much more pronounced in eGFP⁺ cells, compared with eGFP⁻ cells. Moreover, upregulation of transcripts encoding insulin, PDX1, NEUROD1, IAPP, and PC1, occurred preferentially in eGFP⁺ cells (FIGS. 22A-C). These findings demonstrate the redifferentiation potential of dedifferentiated beta cells following ex vivo expansion, and the reproducible differentiating effect of HES1 shRNA in these cells.

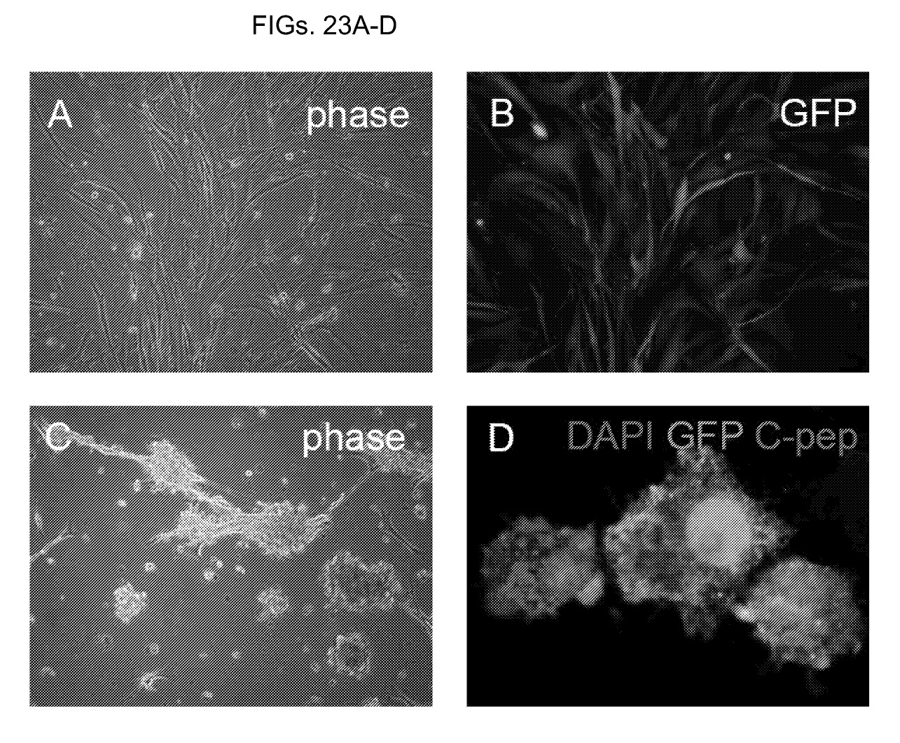

Another approach evaluated for induction of differentiation was shift of the expanded cells to serum-free medium (SFM). Human islet cells from 3 donors were labeled and cultured for 4 weeks under expansion conditions, leading to dedifferentiation. They were then shifted for 8 days to SFM. This shift resulted in activation of insulin mRNA, and appearance of C-peptide staining in 4.7±0.5% of eGFP⁺ cells, compared with <0.1% in cells in the presence of serum (FIGS. 23A-D). Sorted eGFP⁺ cells from 2 donors expanded for 7 weeks in culture showed upregulation of insulin, LAPP and PC1 transcripts following 4-14 days in SFM, while no such upregulation was seen in eGFP⁻ cells. Cell clustering was noted in eGFP⁺ cells (FIGS. 23A-D), but not in eGFP⁻ cells. Thus, the redifferentiation potential of dedifferentiated beta cells following ex vivo expansion was demonstrated by 2 independent approaches. Combination of SFM with HES1 shRNA treatment showed a pronounced additive effect (FIGS. 22A-C).

### Example 5

### Effect of NOTCH-1 Down-Regulation on Insulin Content in B Cells

Materials and Methods

Islet cell culture: As described in Example 1.

Beta cell derived labeled cells at P5 treated for 4 days with 300 nM gamma-secretase inhibitor (S,S)-2-[2-(3,5-difluorophenyl)acetylamino]-N-(5-methyl-6-oxo-6,7-dihydro-5H-dibenzo [b,d]azepin-7-yl)propionamide (DBZ). Control cells were either untreated, or treated with DMSO (solvent). Medium with compound was changed every 48 hours.

RT-PCR analyses: As described for Example 1.

Results

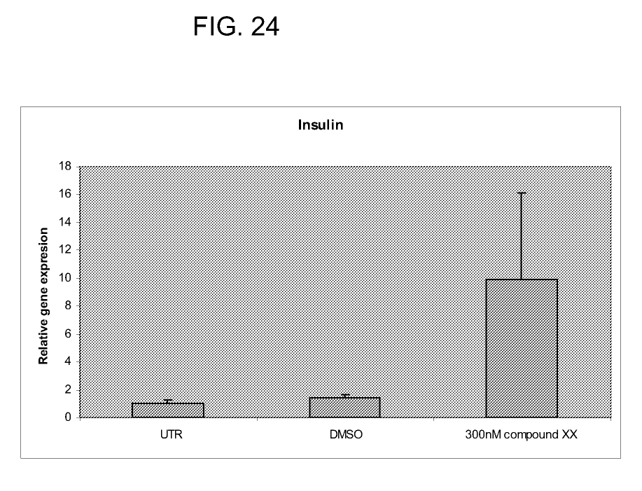

As illustrated in FIG. 24, the gamma secretase inhibitor served to increase insulin expression in the beta cells.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method of generating adult islet beta cells useful for the treatment of diabetes, the method comprising contacting the adult islet beta cells with an agent capable of down-regulating activity and/or expression of at least one component participating in a NOTCH pathway, said component being up-regulated in beta cell dedifferentiation above a predetermined threshold, thereby increasing the insulin content in adult islet beta cells.

2. The method of claim 1, wherein said contacting is effected in a serum-free medium.

3. The method of claim 1, further comprising incubating the adult islet beta cells in a culturing medium, thereby obtaining expanded adult islet beta cells prior to said contacting.

4. The method of claim 1, wherein the increasing is effected in vivo.

5. The method of claim 1, wherein the increasing is effected ex vivo.

6. The method of claim 1, wherein said agent is an oligonucleotide directed to an endogenous nucleic acid sequence expressing said at least one component participating in said NOTCH pathway.

7. The method of claim 6, wherein said at least one component is selected from the group consisting of Hairy and Enhancer of Split 1 (HES1), NOTCH1, NOTCH 2 and NOTCH 3.

8. The method of claim 7, wherein said at least one component is HES1.

9. The method of claim 6, wherein said agent is an siRNA molecule as set forth in SEQ ID NO: 7, SEQ ID NO: 10 or SEQ ID NO: 15.

10. The method of claim 6, wherein said agent is a gamma secretase inhibitor.

11. The method of claim 1, wherein said adult islet beta cells are trypsinized.

12. A method of treating diabetes in a subject, comprising
(a) contacting a population of expanded adult islet beta cells with an agent capable of down-regulating activity and/or expression of at least one component participating in a NOTCH pathway to generate a population of re-differentiated, expanded adult islet beta cells, said component being up-regulated in B cell dedifferentiation above a predetermined threshold; and
(b) transplanting a therapeutically effective amount of said population of re-differentiated, expanded adult islet beta cells into the subject, thereby treating diabetes.

13. A method of purifying a population of dedifferentiated B cells, the method comprising:
(a) permanently tagging primary B cells of cultured human islets, wherein said tagging is irrespective of a subsequent differentiation status of said B cells, to generate a population of permanently tagged B cells;
(b) culturing said permanently tagged B cells under conditions sufficient to allow dedifferentiation of said tagged B cells to generate a population of dedifferentiated tagged B cells; and
(c) isolating said population of dedifferentiated tagged B cells, thereby purifying the population of dedifferentiated B cells.

14. The method of claim 13, wherein said permanently tagging B cells is effected by transfecting said human islets with two expression constructs, wherein a first expression construct comprises a polynucleotide encoding a Cre recombinase polypeptide operatively linked to a B cell specific promoter; and wherein a second expression construct comprises a first polynucleotide encoding a first detectable moiety operatively linked to a constitutive promoter, said first polynucleotide being flanked by LoxP polynucleotides, said second expression construct further comprising a second polynucleotide encoding a second detectable moiety, said second polynucleotide being positioned 3′ to said first polynucleotide.

15. The method of claim 14, wherein said first polynucleotide comprises a nucleic acid sequence as set forth in SEQ ID NO: 11.

16. The method of claim 14, wherein said second polynucleotide comprises a nucleic acid sequence as set forth in SEQ ID NO: 12.

17. An isolated population of primary human dedifferentiated B cells, purified according to the method of claim 13.

18. An isolated population of B cells generated by redifferentiating the isolated population of primary human dedifferentiated cells of claim 17.

19. An isolated population of B cells, comprising a heterologous oligonucleotide capable of down-regulating an activity and/or expression of at least one component participating in a NOTCH pathway.

20. A method of identifying an agent capable of affecting proliferation and/or redifferentiation of dedifferentiated B cells, the method comprising contacting the agent with the isolated population of cells of claim 17 under conditions that allow redifferentiation and/or replication of said dedifferentiated B cells, wherein a change in replication and/or differentiation state of said isolated population of cells is indicative of an agent capable of affecting replication and/or redifferentiation of dedifferentiated B cells.


SEQUENCE LISTING (15 sequences)

SEQ ID NO: 1 - DNA, 28 bp, Artificial sequence
  Single strand DNA oligonucleotide
caccagatctatgcccaagaagaagagg

SEQ ID NO: 2 - DNA, 21 bp, Artificial sequence
  Single strand DNA oligonucleotide
ctcgagctaatcgccatcttc

SEQ ID NO: 3 - DNA, 21 bp, Artificial sequence
  Single strand DNA oligonucleotide
aattcactagtgaacctcttc

SEQ ID NO: 4 - DNA, 20 bp, Artificial sequence
  Single strand DNA oligonucleotide
gatccgatcatattcaataa

SEQ ID NO: 5 - DNA, 18 bp, Artificial sequence
  Single strand DNA oligonucleotide
aacaactccgccccattg

SEQ ID NO: 6 - DNA, 18 bp, Artificial sequence
  Single strand DNA oligonucleotide
ctcctcgcccttgctcac

SEQ ID NO: 7 - DNA, 21 bp, Artificial sequence
  HES1 shRNA target sequence
tggccagtttgctttcctcat

SEQ ID NO: 8 - RNA, 9 nt, Artificial sequence
  Optional ShRNA loop sequence
uucaagaga

SEQ ID NO: 9 - RNA, 9 nt, Artificial sequence
  Optional ShRNA loop sequence
uuuguguag

SEQ ID NO: 10 - DNA, 21 bp, Artificial sequence
  HES1 shRNA target sequence
ccagatcaatgccatgaccta

SEQ ID NO: 11 - DNA, 1527 bp, Artificial sequence
  Rat Insulin promoter fused to CRE recombinase expressing sequence
cgcgtggacacagctatcagtgggaactgtgaaacaacagttcaagggacaaagttactaggtcccccaacaactgcagcctcctggggaatgatgtggaaaaatgctcagccaaggacaaagaaggcctcaccctctctgagacaatgtcccctgctgtgaactggttcatcaggccacccaggagcccctcttaagactctaattaccctaaggctaagtagaggtgttgttgtccaatgagcactttctgcagacctagcaccaggcaagtgtttggaaactgcagcttcagcccctctggccatctgctgatccacccttaatgggacaaacagcaaagtccaggggtcagggggggggtgctttggactataaagctagtggggattcagtaacccccagccctaggatcgccccatcacaagtttgtacaaaaaagcaggctccgcggccgcccccttcaccagatctatgcccaagaagaagaggaaggtgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctggaggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattag

SEQ ID NO: 12 - DNA, 2280 bp, Artificial sequence
  DsRED expressing sequence flanked by two LoxP sites followed by GFP expressing sequence
aagcttgggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgactctagaggatcaattcactagtgaacctcttcgagggacctaataacttcgtatagcatacattatacgaagttatattaagggttccggatcctaagcttccaccggtcgccaccatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgtagcggccagcttggctggacgtaaactcctcttcagacctaataacttcgtatagcatacattatacgaagttatattaagggttattgaatatgatcggatcgctagcggcgcgccatcgatggtaccgtcgactctagaatcgataagcttgtttgatccccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaag

SEQ ID NO: 13 - DNA, 10533 bp, Artificial sequence
  pTripRip400-nlscre-DeltaU3 plasmid sequence
ttgtacaaagtggtgatgggcgggatccggtacctttaagaccaatgacttacaaggcagctgtagatcttagccactttttaaaagaaaaggggggactggaagggctaattcactcccaacgaagacaaaatcgtcgagagatgctgcatataagcagctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattataatagatttagcaggaattgaactaggagtggagcacacaggcaaagctgcagaagtacttggaagaagccaccagagatactcacgattctgcacatacctggctaatcccagatcctaaggattacattaagtttactaacatttatataatgatttatagtttaaagtataaacttatctaatttactattctgacagatattaattaatcctcaaatatcataagagatgattactattatccccatttaacacaagaggaaactgagagggaaagatgttgaagtaattttcccacaattacagcatccgttagttacgactctatgatcttctgacacaaattccatttactcctcaccctatgactcagtcgaatatatcaaagttatggacattatgctaagtaacaaattacccttttatatagtaaatactgagtagattgagagaagaaattgtttgcaaacctgaatagcttcaagaagaagagaagtgaggataagaataacagttgtcatttaacaagttttaacaagtaacttggttagaaagggattcaaatgcataaagcaagggataaatttttctggcaacaagactatacaatataaccttaaatatgacttcaaataattgttggaacttgataaaactaattaaatattattgaagattatcaatattataaatgtaatttacttttaaaaagggaacatagaaatgtgtatcattagagtagaaaacaatccttattatcacaatttgtcaaaacaagtttgttattaacacaagtagaatactgcattcaattaagttgactgcagattttgtgttttgttaaaattagaaagagataacaacaatttgaattattgaaagtaacatgtaaatagttctacatacgttcttttgacatcttgttcaatcattgatcgaagttctttatcttggaagaatttgttccaaagactctgaaataaggaaaacaatctattatatagtctcacacctttgttttacttttagtgatttcaatttaataatgtaaatggttaaaatttattcttctctgagatcatttcacattgcagatagaaaacctgagactggggtaatttttattaaaatctaatttaatctcagaaacacatctttattctaacatcaatttttccagtttgatattatcatataaagtcagccttcctcatctgcaggttccacaacaaaaatccaaccaactgtggatcaaaaatattgggaaaaaattaaaaatagcaatacaacaataaaaaaatacaaatcagaaaaacagcacagtataacaactttatttagcatttacaatctattaggtattataagtaatctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctagtggtttcatttactctaaacctgtgattcctctgaattattttcattttaaagaaattgtatttgttaaatatgtactacaaacttagtagttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccgaattcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggctgatacgcgtggacacagctatcagtgggaactgtgaaacaacagttcaagggacaaagttactaggtcccccaacaactgcagcctcctggggaatgatgtggaaaaatgctcagccaaggacaaagaaggcctcaccctctctgagacaatgtcccctgctgtgaactggttcatcaggccacccaggagcccctcttaagactctaattaccctaaggctaagtagaggtgttgttgtccaatgagcactttctgcagacctagcaccaggcaagtgtttggaaactgcagcttcagcccctctggccatctgctgatccacccttaatgggacaaacagcaaagtccaggggtcagggggggggtgctttggactataaagctagtggggattcagtaacccccagccctaggatcgccccatcacaagtttgtacaaaaaagcaggctccgcggccgcccccttcaccagatctatgcccaagaagaagaggaaggtgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctggaggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattagctcgagaagggtgggcgcgccgacccagctttc

SEQ ID NO: 14 - DNA, 11356 bp, Artificial sequence
  pTrip CMV-loxP-DsRed2-loxP-eGFP DeltaU3 lentiviral vector sequence
aagcttgggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgactctagaggatcaattcactagtgaacctcttcgagggacctaataacttcgtatagcatacattatacgaagttatattaagggttccggatcctaagcttccaccggtcgccaccatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgtagcggccagcttggctggacgtaaactcctcttcagacctaataacttcgtatagcatacattatacgaagttatattaagggttattgaatatgatcggatcgctagcggcgcgccatcgatggtaccgtcgactctagaatcgataagcttgtttgatccccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgggctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaaaatcgtcgagagatgctgcatataagcagctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattataatagatttagcaggaattgaactaggagtggagcacacaggcaaagctgcagaagtacttggaagaagccaccagagatactcacgattctgcacatacctggctaatcccagatcctaaggattacattaagtttactaacatttatataatgatttatagtttaaagtataaacttatctaatttactattctgacagatattaattaatcctcaaatatcataagagatgattactattatccccatttaacacaagaggaaactgagagggaaagatgttgaagtaattttcccacaattacagcatccgttagttacgactctatgatcttctgacacaaattccatttactcctcaccctatgactcagtcgaatatatcaaagttatggacattatgctaagtaacaaattacccttttatatagtaaatactgagtagattgagagaagaaattgtttgcaaacctgaatagcttcaagaagaagagaagtgaggataagaataacagttgtcatttaacaagttttaacaagtaacttggttagaaagggattcaaatgcataaagcaagggataaatttttctggcaacaagactatacaatataaccttaaatatgacttcaaataattgttggaacttgataaaactaattaaatattattgaagattatcaatattataaatgtaatttacttttaaaaagggaacatagaaatgtgtatcattagagtagaaaacaatccttattatcacaatttgtcaaaacaagtttgttattaacacaagtagaatactgcattcaattaagttgactgcagattttgtgttttgttaaaattagaaagagataacaacaatttgaattattgaaagtaacatgtaaatagttctacatacgttcttttgacatcttgttcaatcattgatcgaagttctttatcttggaagaatttgttccaaagactctgaaataaggaaaacaatctattatatagtctcacacctttgttttacttttagtgatttcaatttaataatgtaaatggttaaaatttattcttctctgagatcatttcacattgcagatagaaaacctgagactggggtaatttttattaaaatctaatttaatctcagaaacacatctttattctaacatcaatttttccagtttgatattatcatataaagtcagccttcctcatctgcaggttccacaacaaaaatccaaccaactgtggatcaaaaatattgggaaaaaattaaaaatagcaatacaacaataaaaaaatacaaatcagaaaaacagcacagtataacaactttatttagcatttacaatctattaggtattataagtaatctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctagtggtttcatttactctaaacctgtgattcctctgaattattttcattttaaagaaattgtatttgttaaatatgtactacaaacttagtagttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccgaattcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggggcgat

SEQ ID NO: 15 - DNA, 21 bp, Artificial sequence
  HES1 shRNA target sequence
gaaagtcatcaaagcctatta